(19) United States
(12) Patent Application Publication
(10) Pub. No.: US 2023/0279352 A1
(43) Pub. Date: Sep. 7, 2023

(54) METHODS FOR GENERATING PRIMARY IMMUNE CELLS

(71) Applicant: ASTRAZENECA AB, Södertälje (SE)

(72) Inventors: DEEPALI MALHOTRA, WILMINGTON, DE (US); MICHAEL OVERSTREET, WILMINGTON, DE (US); GORDON MOODY, WILMINGTON, DE (US); MARK COBBOLD, WILMINGTON, DE (US)

(21) Appl. No.: 17/893,445

(22) Filed: Aug. 23, 2022

Related U.S. Application Data
(60) Provisional application No. 63/236,443, filed on Aug. 24, 2021.

(51) Int. Cl. C12N 5/0783; A61K 39/00; C07K 14/705; C07K 14/47

(52) U.S. Cl. CPC C12N 5/0638; A61K 39/4611; A61K 39/4631; C07K 14/705; C07K 14/4747; C12N 2510/04

(57) ABSTRACT

The disclosure relates to methods, cells, and compositions for preparing cell populations and compositions for adoptive cell therapy. In particular, provided herein are methods for expansion and proliferation of primary immune cells including T cell populations.

### CROSS REFERENCE TO RELATED APPLICATIONS

This application claims priority to U.S. Provisional Application No. 63/236,443, filed Aug. 24, 2021, the disclosure of which is incorporated by reference in its entirety.

### FIELD OF THE DISCLOSURE

The disclosure relates to methods, cells, and compositions for preparing cell populations and compositions for adoptive cell therapy. In particular, provided herein are methods for expansion and proliferation of primary immune cells including T cell populations.

### BACKGROUND

Engineered adoptive cellular therapies have been transformative for patients with hematological malignancies in recent years with the first approval for a chimeric antigen receptor (CAR)-based therapy by the FDA in 2017 (Larson & Maus, Nat Rev Cancer 21, 145-161 (2021); Yu, et al., Nature Reviews Drug Discovery 19, 583-584 (2020)). Since 2017, the number of clinical trials investigating adoptive cell therapies such as CAR-T cells, CAR-Natural Killer (NK) and CAR-NKT cells, T cell receptor (TCR)-T cells, tumor infiltrating lymphocytes (TILs), tumor-specific antigen-targeting T cells, and other cellular therapies has grown rapidly. More recently, the first CAR-macrophages (CAR-M) have entered the clinic for the treatment of solid tumors (Mukhopadhyay, Nat Methods 17, 561 (2020); Klichinsky, et al., Nat Biotechnol., 8, 947-953 (2020); Villanueva, Nature Reviews Drug Discovery 19, 308 (2020); ClinicalTrials.gov Identifier: NCT04660929)).

While there is much potential for cellular therapies to be curative for patients, a number of factors limit the widespread development and administration of these drugs. Most cellular therapies are currently produced in an autologous fashion and are associated with variable cell product quality, cytokine release syndrome and other toxicities, extended manufacturing times, high costs, and a limited period in which these therapies may be genetically modified to enhance their efficacy (Larson & Maus, Nat Rev Cancer 21, 145-161 (2021)).

The majority of cellular therapies currently being tested in the clinic utilize CAR-T or CAR-NK cells, as these subsets of immune cells demonstrate potent cytotoxicity. Mature primary human T cells that are used for these therapies are found in the blood and secondary lymphoid organs of humans where they act to protect individuals against infectious diseases and cancer. T cells are comprised of αβ (“classic” T cells) and γδ subsets. αβ T cells consist of CD4⁺ helper T cells and CD8⁺ cytotoxic T cells. CD4⁺ T cells can be further subdivided into TH1 cells, TH2 cells, TH9 cells, TH17 cells, TFH cells, and regulatory T cells. Many αβ T cell subsets exhibit potent cytotoxic function which has been harnessed for the development of cellular therapies.

Similarly, mature primary human NK cells that can be used for cellular therapies are found in the blood, secondary lymphoid organs, liver, and mucosal associated lymphoid tissues, sites that NK cells patrol for the presence of pathogens or transformed cells (Jianhua, et al., Trends in Immunology 34, 573-582 (2013). Like T cells, NK cells demonstrate potent cytotoxic function and are of interest for the development of cellular therapies.

However, primary human immune cells, such as T cells and NK cells, also possess a finite potential for proliferation in vitro and in vivo, limiting their ability to be used for the generation of widespread off-the-shelf cellular therapies. Further, this limited proliferative capacity of mature primary human immune cells impairs their ability to be genetically edited to mitigate cytokine release syndrome and other potential cellular-therapy-associated toxicities, to overcome tumor microenvironment-associated challenges, and to prevent the rejection of allogeneic cellular therapy products in patients.

Patient-derived leukemic cell lines have been studied for decades in cell culture, where their transformed status confers a long-term proliferative capacity that enables their use in a variety of cellular assays. This in turn has facilitated the development of numerous therapies. These cells, however, generally lack the potent cytotoxic function of mature primary human T and NK cells, as they are often immature or derived from dysfunctional T cell clones. The transformed nature of these cells can be mapped on to a collection of mutations that are also frequently found in patients with T cell acute lymphoblastic leukemia. Furthermore, mature T cells from non-human primates can be transformed by herpes viruses through pathways that converge on some of the same mechanisms involved in the transformation of primary human T cells in patients (Biesinger, et al., Proc Natl Acad Sci USA 89, 3116-3119 (1992); Weber, et al., Proc Natl Acad Sci USA 90, 11049-11053 (1993); Fickenscher H, Fleckenstein B., Philos Trans R Soc Lond B Biol Sci. 356(1408):545-67 (2001); Tsygankov, J Cell Physiol. 203(2):305-18 (2005).

While previous studies suggest that primary human T cells may be immortalized through the over-expression of factors such as telomerase-reverse transcriptase (TERT) (Barsov, Methods Mol Biol. 511, 143-58 (2009); Rufer, et al., Blood 98, 597-603 (2001); Hooijberg, et al., J Immunol. 165, 4239-45 (2000)) and human T cell leukemia virus type 1 or human T cell leukemia virus type 2 (HTLV-1/HTLV-2) transcriptional trans-activator protein Tax (Akagi, et. al., Oncogene 14, 2071-2080 (1997); Grassmann, et al., Proc Natl Acad Sci USA 86, 3351-3355 (1989); Ren, et al., J Biol. Chem. 287, 34683-34693 (2012), or by viruses such as Herpesvirus saimiri (Biesinger, et al., Proc Natl Acad Sci USA 89, 3116-3119 (1992); Weber, et al., Proc Natl Acad Sci USA 90, 11049-11053 (1993)) and HTLV-1/HTLV-2, these approaches are not highly reproducible and can result in reprogramming of modified or infected cells. In addition, cells whose proliferative longevity has been enhanced through the overexpression of TERT still require the use of feeder cells or extensive exogenous stimulation through their T cell receptors to drive proliferation (Rufer, et al., Blood 98, 597-603 (2001); Hooijberg, et al., J Immunol. 165, 4239-45 (2000)). The use of allogeneic feeder cells and extensive repeat stimulation is undesirable when establishing a bank of mature primary human T or NK cells as these methodologies are challenging to scale and may ultimately drive the cells to a dysfunctional state. Further, the use of infectious agents with the ability to transform mature primary human T or NK cells limits the use of these cells in the development of cellular therapies as patients are often immunocompromised.

In light of these challenges, there is a significant need to establish alternative methods by which to extend the proliferative longevity of primary human immune cells to enable large-scale manufacturing of allogeneic cytotoxic cells. The disclosure describes methods and cells that address this unmet need.

### SUMMARY

The disclosure herein provides a method of generating a population of primary immune cells resistant to replicative senescence (RRS), comprising: (a) introducing one or more genetic edits to primary immune cells; and (b) culturing the primary immune cells in a culture medium; wherein the culturing induces proliferation of the primary immune cells to yield a population of primary immune cells resistant to replicative senescence (RRS).

The disclosure herein also provides a method of generating a population of primary immune cells resistant to replicative senescence (RRS), comprising: (a) inhibiting the expression of one or more endogenous regulatory factors in the primary immune cells, wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP); (b) inhibiting the expression of one or more endogenous immune related genes in the primary immune cells, wherein the endogenous immune related gene is beta-2 microglobulin (B2M), and/or T-cell receptor α constant (TRAC); and (c) culturing the primary immune cells in a culture medium; wherein the culturing induces proliferation of the primary immune cells to yield a population of primary immune cells resistant to replicative senescence (RRS).

The disclosure herein further provides a method of generating a population of primary immune cells resistant to replicative senescence (RRS), comprising: (a) inhibiting the expression of one or more endogenous regulatory factors in the primary immune cells, wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP); (b) inhibiting the expression of one or more endogenous immune related genes in the primary immune cells, wherein the endogenous immune related gene is beta-2 microglobulin (B2M), and/or T-cell receptor α constant (TRAC); and (c) culturing the primary immune cells in a culture medium; wherein the culturing induces proliferation of the primary immune cells to yield a population of primary immune cells resistant to replicative senescence (RRS).

The disclosure herein provides a method of generating a population of primary immune cells resistant to replicative senescence (RRS), comprising: (a) inhibiting expression of one or more endogenous regulatory factors in the primary immune cells, wherein endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP); and (b) culturing the primary immune cells in a culture medium; wherein the culturing induces proliferation of the primary immune cells to yield a population of primary immune cells resistant to replicative senescence (RRS).

The disclosure herein also provides an engineered immune cell population produced according to the described methods. The disclosure herein further provides a pharmaceutical composition comprising the aforementioned engineered immune cell population and a pharmaceutically acceptable carrier. The disclosure herein further provides a method of treating a cancer in a subject in need thereof, comprising administering to the subject a therapeutically effective amount of the aforementioned pharmaceutical composition.

The disclosure herein provides an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), and/or T-cell receptor a constant (TRAC).

The disclosure herein provides an engineered T cell expressing a transgene encoding a B-cell lymphoma-extra large (Bcl-XL), wherein the engineered T cell does not express cyclin-dependent kinase inhibitor 2A (CDKN2A), cyclin-dependent kinase inhibitor 2B (CDKN2B), S-methyl-5′-thioadenosine phosphorylase (MTAP), and/or phosphatase and tensin homolog (PTEN).

### BRIEF DESCRIPTION OF THE DRAWINGS

FIGS. 1A-1B illustrate that Bcl-xL insertion conferred a selective advantage for T cell survival in long-term culture. Total primary human T cells (FIG. 1A) or purified primary human CD8⁺ T cells (FIG. 1B) were isolated, stimulated, transfected, and restimulated as described in FIG. 2.

FIG. 2 illustrates the method for identifying survival-enhancing transgenes in primary human T cells.

FIGS. 3A-3B illustrate that ablation of expression of cell cycle regulatory molecules enhanced the proliferative capacity of T cells in long-term culture.

FIGS. 4A-4D illustrate that restimulation of T_{REX}+Bcl-xL cells can enhance their proliferation in long-term culture. FIG. 4A shows total fold expansion of T_{REX}+Bcl-xL cells over time. FIGS. 4B-4D show total fold expansion of T_{REX}+Bcl-xL cells and PTEN-deficient T_{REX}+Bcl-xL cells that were restimulated with αCD3 or αCD3/αCD28 Dynabeads for 3 days after which cells were debeaded. Total fold expansion was tracked and graphed for resting and treated cells over time. Arrows indicate periods of restimulation. Black arrow indicates timepoint of evaluation of additional restimulation modalities. FIGS. 4A-4D show log scale.

FIGS. 5A-5B illustrate that T_{REX}+Bcl-xL cells are dependent on IL-2 for expansion and survival in cell culture. Total fold expansion (FIG. 5A) and cell viability (FIG. 5B) were assessed for 3 T_{REX}+Bcl-xL lines established as in FIG. 3 from two different donors grown in the presence of increasing quantities of recombinant human interleukin 2 (IL-2) for 6 days.

FIGS. 6A-6K illustrate that T_{REX}+Bcl-xL cells phenotypically resemble normal primary human CD8⁺ T cells. T_{REX}+Bcl-xL cells were stained with a fixable viability dye as well as a panel of antibodies to CD3, CD4, CD8, CD28, CD45RO, CCR7, PD1, and TIGIT. T_{REX}+Bcl-xL lines demonstrate expression of CD3 (FIG. 6A) and comprise a high frequency of CD8⁺ cells (FIG. 6B). T_{REX}+Bcl-xL lines exhibited donor or cell-line specific attributes as exemplified by expression of markers such as PD1 and TIGIT (FIG. 6C), CD28 (FIG. 6D), and CCR7 and CD45RO (FIG. 6E) regardless of Bcl-xL overexpression (GFP⁺ and GFP⁻ cells). T_{REX}+Bcl-xL lines demonstrate expression of CCR2 (FIG. 6F), CCR5 (FIG. 6G), and CXCR3 (FIG. 6J). Expression of CCR6 (FIG. 6H) was heterogenous, while expression of CCR7 (FIG. 6I) and CXCR5 (FIG. 6K) was low to absent.

FIGS. 7A-7F illustrate that T_{REX}+Bcl-xL cells are cytotoxic. Percent cytolysis was computed 12 hours (FIG. 7A) and 24 hours (FIG. 7B) post-addition of effector cells and a T cell engager or control antibody. Supernatants were collected from co-cultures 72 hours post-addition of effector cells and the T cell engager and analyzed for the presence of interferon γ (IFN-γ) (FIG. 7C), IL-2 (FIG. 7D), tumor necrosis factor α (TNF-α) (FIG. 7E), and granzyme B (FIG. 7F).

FIGS. 8A-8G illustrate that T_{REX}+Bcl-xL cells can produce functional CAR-T_{REX}+Bcl-xL cells. Surface CAR expression was assessed 22-days post-transduction using flow cytometry (FIG. 8A). Percent cytolysis was computed 12 hours (FIG. 8B) and 24 hours (FIG. 8C) after addition of effector cells. CAR-T_{REX} activity was benchmarked against that of CAR-T cells and CAR-CD8⁺ T cells. Supernatants were collected from co-cultures 72 hours post-addition of effector cells and analyzed for the presence of IFN-γ (FIG. 8D), IL-2 (FIG. 8E), TNF-α (FIG. 8F), and granzyme B (FIG. 8G).

FIGS. 9A-9C show that T_{REX} cells traffic to similar locations as primary CD8⁺ T cells and are responsive to IL-2 in vivo.

FIGS. 10A-10B show that CAR-T_{REX} cells respond to IL-2 and IL-15 in vivo.

FIGS. 11A-11D show that CAR-T_{REX} cells target solid tumors in vivo.

FIG. 12 shows REX edits reproducibility confer enhanced in vitro proliferation relative to unmodified donor-matched CD8⁺ T cells. Fold expansion of T_{REX} cells or donor-matched primary (unedited) CD8⁺ T cells was tracked over time for 4 additional healthy donors.

FIGS. 13A and 13B show that CAR-T_{REX} cells target BCMA+ tumor cells similarly to unmodified CAR-T cells.

FIG. 14 shows that anti-BCMA-T_{REX} and anti-HER2-T_{REX} cells produce lower levels of inflammatory cytokines than anti-BCMA-CAR-T cells and anti-HER2-CAR-T cells following CAR engagement.

FIG. 15 shows that CAR-T_{REX} cells target BCMA+ tumor cells, persist in a serial kill assay, and respond to IL-2.

FIG. 16 shows that the T_{REX} cell phenotype can be generated using different combinations of edits.

FIG. 17 shows that the T_{REX} cells are edited at the expected loci.

FIGS. 18A, 18B, and 18C show that T_{REX} cells demonstrate enrichment in cell cycle-associated gene signatures.

FIG. 19 shows T_{REX} cells are dependent on IL-2 for survival and proliferation.

FIGS. 20A and 20B show that CAR-T_{REX} cells target HER2hi tumor cells similarly to unmodified CAR-T cells with less overall cytokine production.

FIG. 21 shows that REX edits bolster the proliferative capacity of CD4+T_{REX} cells.

FIG. 22 shows that γδ T_{REX} cells can be generated using REX edits.

FIG. 23 shows that γδ T_{REX} cells are active in a T cell engager (TCE) assay in vitro.

FIG. 24 shows that γδ T_{REX} cells can be generated from multiple γδ T cell subsets and diversity is maintained following CAR transduction.

FIGS. 25A and 25B show that γ6-T_{REX} cells target BCMA+ tumor cells similarly to unmodified CAR-T cells.

FIG. 26 shows that REX edits in NK cells support an NK_{REX} cell phenotype.

FIG. 27 shows that NK_{REX} cells are dependent on cytokines for proliferation and survival.

FIG. 28 shows that NK_{REX} cells maintain CAR expression over time.

FIGS. 29A-29C show that NK_{REX} cells are cytotoxic in vitro and CAR expression can further enhance potency.

FIG. 30 shows that T_{REX} cells are sensitive to T cell depleting agents and chemotherapies.

FIG. 31 shows that B2MKO T_{REX} cells are sensitive to NK cell mediated depletion and this can be modulated using anti-CD38 antibodies.

### DETAILED DESCRIPTION

The disclosure relates to methods, cells, and compositions for preparing cell populations and compositions for adoptive cell therapy. In particular, provided herein are methods for expansion and proliferation of primary immune cells including T cell populations.

As utilized in accordance with the present disclosure, unless otherwise indicated, all technical and scientific terms shall be understood to have the same meaning as commonly understood by one of ordinary skill in the art. Unless otherwise required by context, singular terms shall include pluralities and plural terms shall include the singular.

As used herein, the terms “comprise” and “include” and variations thereof (e.g., “comprises,” “comprising,” “includes,” and “including”) will be understood to indicate the inclusion of a stated component, feature, element, or step or group of components, features, elements or steps but not the exclusion of any other component, feature, element, or step or group of components, features, elements, or steps. Any of the terms “comprising,” “consisting essentially of,” and “consisting of” may be replaced with either of the other two terms, while retaining their ordinary meanings.

As used herein, the singular forms “a,” “an” and “the” include plural referents unless the context clearly indicates otherwise.

Percentages disclosed herein can vary in amount by ±10, 20, or 30% from values disclosed and remain within the scope of the contemplated disclosure.

Unless otherwise indicated or otherwise evident from the context and understanding of one of ordinary skill in the art, values herein that are expressed as ranges can assume any specific value or sub-range within the stated ranges in different aspects of the disclosure, to the tenth of the unit of the lower limit of the range, unless the context clearly dictates otherwise.

As used herein, ranges and amounts can be expressed as “about” a particular value or range. The term “about” also includes the exact amount. For example, “about 5%” means “about 5%” and also “5%.” The term “about” can also refer to ±10% of a given value or range of values. Therefore, about 5% also means 4.5%-5.5%, for example. Unless otherwise clear from context, all numerical values provided herein are modified by the term “about.”

As used herein, the terms “or” and “and/or” can describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” Resistant to replicative senescence (RRS) refers to primary immune cells that are resistant to replicative senescence (RS) that leads to a finite number of population doublings. As a result, the population of primary immune cells described herein advantageously have extended proliferative capacity.

In one aspect the disclosure herein provides a method of generating a population of primary immune cells resistant to replicative senescence (RRS), comprising: i) introduction of one or more genetic edits into the primary immune cells; and ii) culturing the primary immune cells in a culture medium; wherein the culturing induces proliferation of the primary immune cells to yield a population of primary immune cells resistant to replicative senescence (RRS).

In one aspect the disclosure herein provides a method of generating a population of primary immune cells resistant to replicative senescence (RRS), comprising: i) introducing a transgene encoding B-cell lymphoma-extra large (Bcl-xL) into the primary immune cells; ii) inhibiting the expression of one or more endogenous regulatory factors selected from cyclin-dependent kinase inhibitor 2A (CDKN2A), cyclin-dependent kinase inhibitor 2B (CDKN2B), and S-methyl-5′-thioadenosine phosphorylase (MTAP) in the primary immune cells; and iii) culturing the primary immune cells in a culture medium; wherein the culturing induces proliferation of the primary immune cells to yield a population of primary immune cells resistant to replicative senescence (RRS). In some aspects, the method comprises inhibiting the expression of one or more endogenous immune related genes in the primary immune cells. In certain aspects, the endogenous immune related gene is beta-2 microglobulin (B2M), or T-cell receptor α constant (TRAC). In some aspects, the method comprises inhibiting the expression of CD38.

In one aspect the disclosure herein provides a method of generating a population of primary immune cells resistant to replicative senescence (RRS), comprising: i) inhibiting the expression of one or more endogenous regulatory factors selected from cyclin-dependent kinase inhibitor 2A (CDKN2A), cyclin-dependent kinase inhibitor 2B (CDKN2B), and S-methyl-5′-thioadenosine phosphorylase (MTAP) in primary immune cells; and ii) culturing the primary immune cells in a culture medium; wherein the culturing induces proliferation of the primary immune cells to yield a population of primary immune cells resistant to replicative senescence (RRS). In some aspects, the method comprises inhibiting the expression of one or more endogenous immune related genes in the primary immune cells. In certain aspects, the endogenous immune related gene is beta-2 microglobulin (B2M), or T-cell receptor α constant (TRAC). In some aspects, the method comprises inhibiting the expression of CD38.

A genetic edit refers to a change to the genetic material of the primary immune cell. A genetic edit includes genetic material to be added, removed, or altered. In particular aspects, genetic edits comprise introducing a transgene into the primary immune cells and/or inhibiting the expression of a gene in the primary immune cell. In particular aspects, introducing one or more genetic edits comprise introducing one or more transgenes encoding an anti-apoptotic factor or a virally-derived factor into the primary immune cells.

The term “Primary immune cell(s)” can refer to any cell(s) involved in a primary immune response such as T cells, B-cells and NK cells, neutrophils, and monocytes/macrophages/dendritic cells. In some aspects, primary immune cells can comprise total T cells, CD4-positive T cells, CD8-positive T cells, regulatory T cells, gamma-delta T cells, mucosal associated invariant T (MAIT) T cells, natural killer (NK) cells, or natural killer T (NKT) cells.

The term “transgene” refers to any nucleic acid sequence that is introduced into the cell by experimental manipulations. A transgene may be an “endogenous DNA sequence” or a “heterologous DNA sequence.” The transgene may be isolated and obtained in suitable quantity using one or more methods that are well known in the art. These methods and others useful for isolating a transgene are set forth, for example, in Sambrook et al. (supra) and in Berger and Kimmel (Methods in Enzymology: Guide to Molecular Cloning Techniques, vol. 152, Academic Press, Inc., San Diego, Calif. (1987)).

The transgene can be incorporated into a “transgene construct” that comprises the gene of interest along with other regulatory DNA sequences needed either for temporal, or cell specific, or enhanced expression of the transgenes of interest.

The transgene may be introduced into the cells by any suitable method or technique known in the art. In particular aspects, the transgene is introduced using a plasmid-based DNA transposon, lentivirus platform, or site-specific integration via CRISPR. The transgene expression in the cell can be constitutive or inducible.

In particular aspects, the transgene encodes an anti-apoptotic factor. An “anti-apoptotic factor” refers to a protein or an oligonucleotide (which may be an oligonucleotide encoding for a protein or a silencing nucleotide) which acts to prevent apoptosis of a cell, in particular a cell experiencing stress, a cell received signal to undergo apoptosis or a cell undergoing abnormal cell proliferation. In particular aspects, the anti-apoptotic factor is B-cell lymphoma-extra large (Bcl-xL) or B-cell lymphoma 2 (Bcl-2).

In particular aspects, the transgene encodes a virally-derived factor. A “virally-derived factor” refers to both naturally-occurring viral peptides, polypeptides, or proteins, as well as peptides, polypeptides, or proteins displaying a degree of sequence identity and/or similarity to a viral protein and/or maintaining one or more structural, mechanistic, or antigenic qualities of the viral protein. In particular aspects, the virally-derived factor is from Saimiriine gammaherpesvirus 2 StpA All, Herpesvirus saimiri StpC, Herpesvirus saimiri Tip, or a modified Herpesvirus ateles-Epstein-Barr virus Tio-LMP1.

In other aspects, the transgene encodes a protein relating to activating signals in the cell.

In some aspects, methods of the disclosure further include inhibiting the expression of one or more endogenous regulatory factors in the primary immune cells such that the activity of the endogenous regulatory factor is eliminated or reduced. As used herein a “regulatory factor” refers to a gene that encodes a protein involved in regulating the cell cycle arrest, cell death, or signal suppression. The endogenous regulatory factor may be down regulated or blocked by any suitable method or technique known in the art. Known methods for down regulation of gene expression or decreasing the activity of a factor include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous regulatory factor can be complete inhibition, partial inhibition, down regulation of gene expression or decreasing the activity of a factor. In some aspects, endogenous regulatory factor activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). A regulatory factor includes a gene that encodes a protein involved in regulating the cell cycle arrest, cell death or signal suppression. In particular aspects, the one or more endogenous regulatory factors are cyclin-dependent kinase inhibitor 2A (CDKN2A), cyclin-dependent kinase inhibitor 2B (CDKN2B), and/or S-methyl-5′-thioadenosine phosphorylase (MTAP). In particular aspects, the one or more endogenous regulatory factors are RB Transcriptional Corepressor 1 (RB1), TP53, Autophagy and Beclin 1 Regulator 1 (AMBRA1), Neurofibromatosis type 1 (NF1), Tyrosine-protein phosphatase non-receptor type 2 (PTPN2), or Suppressor of Cytokine Signaling 1 (SOCS1).

The term “endogenous” refers to developing or originating within a cell, a tissue, or an organism or part of a cell, a tissue or an organism.

In some aspects, methods of the disclosure further include inhibiting the expression of one or more endogenous immune related genes in the primary immune cells such that the activity of the immune related genes is eliminated or reduced. As used herein an “immune related gene” refers to a gene that encodes a protein involved in effecting an immune response. In certain aspects, the immune related gene encodes a protein that is involved in host-versus-graft (HvG) and graft-versus-host (GvH) allogeneic immune responses. The immune related gene may be down regulated or blocked by any suitable method or technique known in the art. Known methods for down regulation of gene expression or decreasing the activity of an immune related gene include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. The inhibition of the endogenous immune related gene can be complete inhibition, partial inhibition, down regulation of gene expression or decreasing the activity of a factor. In some aspects, endogenous immune related gene activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%). An immune related gene includes a gene that encodes a protein involved in effecting an immune response. An immune related gene can encode a protein that is involved in host-versus-graft (HvG) and graft-versus-host (GvH) allogeneic immune responses. In particular aspects, the one or more endogenous immune related genes are beta-2 microglobulin (B2M), or T-cell receptor α constant (TRAC). In particular aspects, the one or more endogenous immune related genes are genes of the major histocompatibility complex (MHC), human leukocyte antigen class I genes (e.g. HLA-A, HLA-B, HLA-C), human leukocyte antigen class II genes (HLA-DR, HLA-DQ, and HLA-DP), T cell receptors (e.g. αβ T cell receptor), interleukin 1 (IL-1), interleukin 2 (IL-2), interleukin 4 (IL-4), interleukin 6 (IL-6), interleukin 10 (IL-10), interleukin 23 (IL-23), interferon-γ (IFNγ), CCL2, CCL3, CCL4, CCL5, CXCL2, CXCL9-11, CCL17, CCL27, programmed death-1 (PD-1), TIM3, or TIGIT.

In further aspects, the methods disclosed herein include inhibiting the expression of cluster of differentiation 38 (CD38) in the primary immune cells such that the activity of CD38 is eliminated or reduced. CD38 may be down regulated or blocked by any suitable method or technique known in the art. Known methods for down regulation of gene expression or decreasing the activity of CD38 include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some aspects, CD38 activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

In further aspects, the methods disclosed herein include inhibiting the expression of phosphatase and tensin homolog (PTEN) in the primary immune cells such that the activity of PTEN is eliminated or reduced. PTEN may be down regulated or blocked by any suitable method or technique known in the art. Known methods for down regulation of gene expression or decreasing the activity of PTEN include, but are not limited to, CRISPR/Cas (including cytosine and adenine base editors), microRNA, shRNA, RNAi, TALENs, zinc finger nucleases, meganucleases, neutralizing antibodies, small molecule inhibitors, chemical inhibitors blocking downstream signaling pathways, and the like. In some aspects, PTEN activity or gene expression is reduced by between 1%-100% (i.e., 1%, 5%, 10%, 15%, 20%, 25%, 30%, 35%, 40%, 45%, 50%, 55%, 60%, 65%, 70%, 75%, 80%, 85%, 90%, 95%, 98%, 99%, 100%).

The term “T_{REX}” refers to a “T cell that is Renewably EXpandable” using e.g., the techniques and genetic modifications provided herein. More specifically, T_{REX} cells refer to cells with decreased or ablated expression of some or all of cyclin-dependent kinase inhibitor 2A (CDKN2A), cyclin-dependent kinase inhibitor 2B CDKN2B, and S-methyl-5′-thioadenosine phosphorylase (MTAP).

In some aspects, inhibiting the expression of one or more endogenous regulatory factors occurs after introduction of the one or more transgene into the cells. In some aspects, primary immune cells in which one or more transgene has been introduced are cultured for at least 2 days, at least 5 days, at least 10 days, at least 11 days, at least 12 days, at least 13 days, at least 14 days, at least 15 days, at least 16 days, at least 17 days, at least 18 days, at least 19 days, at least 20 days before inhibition of one or more endogenous regulatory factor is performed. In further aspects, inhibiting the expression of PTEN occurs after introduction of the one or more transgenes into the cells. In some aspects, the method comprises the following sequential steps i) introducing one or more transgenes into the immune cells and then culturing the cells for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; ii) inhibiting one or more endogenous regulatory factor culturing the cell for at least 2 days, 5 days, at least 10 days, at least 11 days, at least 12 days, at least 13 days, at least 14 days, at least 15 days, at least 16 days, at least 17 days, at least 18 days, at least 19 days, at least 20 days; and iii) inhibiting PTEN expression.

Primary immune cells are cultured under conditions appropriate for promoting proliferation and expansion. In vitro expansion using the culture step activates and induces proliferation of the primary immune cells to yield an expanded population comprising primary immune cells sufficient in numbers for use in therapy.

The methods disclosed herein are performed ex vivo meaning that the methods take place outside an organism. Treatment of immune cells ex vivo means exposing cells to certain biological molecules in vitro preferably under sterile conditions. In some cases, ex vivo methods additionally include culturing immune cells that have been isolated from a human prior to administration back into the same or different human subject.

The primary immune cells including the expanded populations, and/or the engineered T cells of this disclosure can comprise total T cells, CD4-positive T cells, CD8-positive T cells, regulatory T cells, gamma-delta T cells, mucosal associated invariant T (MAIT) T cells, natural killer (NK) cells, or natural killer T (NKT) cells. T cells are broadly divided into cells expressing CD4 on their surface (also referred to as CD4-positive cells) and cells expressing CD8 on their surface (also referred to as CD8-positive cells). T cells appropriate for use according to the methods provided herein are mononuclear lymphocytes derived from bone marrow (BM), peripheral blood (PB), or cord blood (CB) of a human donor. These cells could be collected directly from BM, PB, or CB or after mobilization or stimulation via administration of growth factors and/or cytokines such as granulocyte-colony stimulating factor (G-CSF) or granulocyte-macrophage colony-stimulating factor (GM-CSF) to allogeneic or autologous donors. Those skilled in the art would appreciate that there are many established protocols for isolating peripheral blood mononuclear cells (PBMC) from peripheral blood. Isolation of PBMC can be aided by density-gradient separation protocols, usually employing a density-gradient centrifugation technique using Ficoll®-Hypaque or Histopaque® for separating lymphocytes from other elements in the blood. Preferably, PBMC isolation is performed under sterile conditions. Isolation of PBMC can also use negative selection kits. Alternatively, cell elutriation methods may be employed to separate mononuclear cell populations. In some aspects, the primary immune cells are human.

In some cases, methods of this disclosure further include introducing a genetically engineered or chimeric antigen receptor into activated T cells, wherein the method thereby generates an expanded population comprising of T cells expressing the genetically engineered or chimeric antigen receptor. Chimeric antigen receptors (CARs), also known as chimeric T cell receptors, artificial T cell receptors, and chimeric immunoreceptors, are engineered receptors, which graft specificity onto an immune effector cell. In general, a chimeric antigen receptor is a transmembrane protein having a target-antigen binding domain that is fused via a spacer and a transmembrane domain to a signaling endodomain. When the CAR binds its target antigen, an activating signal is transmitted to the T cell. In one embodiment, a polynucleotide that encodes a chimeric antigen receptor is introduced to the primary cells. In one embodiment, a nucleic acid vector encoding the chimeric antigen receptor or genetically engineered receptor is introduced into the T cells whereby the T cells express the chimeric antigen receptor. In some aspects, the CAR binds glypican 3 (GPC3), human epidermal growth factor receptor 2 ((HER2); also known as Erb-B2 Receptor Tyrosine Kinase 2 (ERBB2)), B-cell maturation antigen (BCMA). In certain aspects, the CAR can bind any target for use in immunotherapy.

CAR construct design: CAR constructs of the present disclosure can have several components, many of which can be selected based upon a desired or refined function of the resultant CAR construct. In addition to an antigen binding domain, CAR constructs can have a spacer domain, a hinge domain, a signal peptide domain, a transmembrane domain, and one or more costimulatory domains. Selection of one component over another (i.e., selection of a specific co-stimulatory domain from one receptor versus a co-stimulatory domain from a different receptor) can influence clinical efficacy and safety profiles.

Antigen binding domain: Antigen binding domains contemplated herein can include antibodies or one or more antigen-binding fragments thereof. In an embodiment, a CAR construct targets GPC3. In an embodiment, a CAR construct targets BCMA. In an embodiment, a CAR construct targets HER2. In an embodiment, a CAR construct targets any molecule useful in an immunotherapy. In certain aspects, the antigen binding domain comprises a single chain variable fragment (scFv) containing light and heavy chain variable regions from one or more antibodies specific for GPC3, BCMA, or HER2 that are either directly linked together or linked together via a flexible linker (e.g., a repeat of G4S having 1, 2, 3 or more repeats).

Spacer domain: A CAR construct can have a spacer domain to provide conformational freedom to facilitate binding to the target antigen on the target cell. The optimal length of a spacer domain may depend on the proximity of the binding epitope to the target cell surface. For example, proximal epitopes can require longer spacers and distal epitopes can require shorter ones. Besides promoting binding of the CAR to the target antigen, achieving an optimal distance between a CAR cell and a cancer cell may also help to sterically occlude large inhibitory molecules from the immunological synapse formed between the CAR cell and the target cancer cell. A CAR can have a long spacer, an intermediate spacer, or a shorter spacer. Long spacers can include a CH2CH3 domain (˜220 amino acids) of immunoglobulin G1 (IgG1) or IgG4 (either native or with modifications common in therapeutic antibodies, such as a S228P mutation), whereas the CH3 region can be used on its own to construct an intermediate spacer (˜120 amino acids). Shorter spacers can be derived from segments (<60 amino acids) of CD28, CD8α, CD3 or CD4. Short spacers can also be derived from the hinge regions of IgG molecules. These hinge regions may be derived from any IgG isotype and may or may not contain mutations common in therapeutic antibodies such as the S228P mutation mentioned above.

Hinge domain: A CAR can also have a hinge domain. The flexible hinge domain is a short peptide fragment that provides conformational freedom to facilitate binding to the target antigen on the tumor cell. It may be used alone or in conjunction with a spacer sequence. The terms “hinge” and “spacer” are often used interchangeably—for example, IgG4 sequences can be considered both “hinge” and “spacer” sequences (i.e., hinge/spacer sequences).

Signal peptide: A CAR construct can further include a sequence comprising a signal peptide. Signal peptides function to prompt a cell to translocate the CAR to the cellular membrane. Examples include an IgG1 heavy chain signal polypeptide, Ig kappa or lambda light chain signal peptides, granulocyte-macrophage colony stimulating factor receptor 2 (GM-CSFR2 or CSFR2) signal peptide, a CD8a signal polypeptide, or a CD33 signal peptide.

Transmembrane domain: A CAR construct can further include a sequence comprising a transmembrane domain. The transmembrane domain can include a hydrophobic a helix that spans the cell membrane. The properties of the transmembrane domain have not been as meticulously studied as other aspects of CAR constructs, but they can potentially affect CAR expression and association with endogenous membrane proteins. Transmembrane domains can be derived, for example, from CD4, CD8α, or CD28.

Costimulatory domain: A CAR construct can further include one or more sequences that form a co-stimulatory domain. A co-stimulatory domain is a domain capable of potentiating or modulating the response of immune effector cells. Co-stimulatory domains can include sequences, for example, from one or more of CD3zeta (or CD3z), CD28, 4-1BB, OX-40, ICOS, CD27, GITR, CD2, IL-2R13 and MyD88/CD40. The choice of co-stimulatory domain influences the phenotype and metabolic signature of CAR cells. For example, CD28 co-stimulation yields a potent, yet short-lived, effector-like phenotype, with high levels of cytolytic capacity, interleukin-2 (IL-2) secretion, and glycolysis. By contrast, T cells modified with CARs bearing 4-1BB costimulatory domains tend to expand and persist longer in vivo, have increased oxidative metabolism, are less prone to exhaustion, and have an increased capacity to generate central memory T cells.

In particular aspects, the methods disclosed herein compromise early stimulation of the primary immune cells to ensure the cells are in cycle prior to introduction of the one or more genetic edits to the cells. In other aspects, the methods disclosed herein compromise late stimulation of the primary immune cells (also referred to as “restimulation”). Once the primary immune cells have exited cell cycle, the cells are restimulated causing the cells to re-enter into cell cycle (i.e., proliferation).

In particular aspects, the methods disclosed herein further comprise stimulating the primary immune cells before the introduction of the one or more genetic edits to the primary immune cells. In particular aspects, the primary immune cells are stimulated before at least 1 day, at least 2 days, at least 5 days, at least 10 days, at least 15 days, at least 20 days, or at least 30 days before the introduction of the one or more genetic edits to the primary immune cell. Thus, in particular aspects, provided herein is a method of generating a population of primary immune cells resistant to replicative senescence (RRS), comprising: i) stimulating the primary immune cells; ii) introducing one or more genetic edits into the primary immune cells; iii) culturing the primary immune cells in a culture medium; wherein the culturing induces proliferation of the primary immune cells to yield a population of primary immune cells resistant to replicative senescence (RRS).

In particular aspects, the methods disclosed herein further comprise stimulating the primary immune cells following the introduction of the one or more genetic edits to the primary immune cells. In particular aspects, the primary immune cells are stimulated after at least 1 day, at least 2 days, at least 5 days, at least 10 days, at least 15 days, at least 20 days, or at least 30 days following the introduction of the one or more genetic edits to the primary immune cell. Thus, in particular aspects, provided herein is a method of generating a population of primary immune cells resistant to replicative senescence (RRS), comprising: i) introducing one or more genetic edits into the primary immune cells; ii) culturing the primary immune cells in a culture medium; iii) stimulating the primary immune cells; and iv) culturing the primary immune cells in the culture medium; wherein the culturing induces proliferation of the primary immune cells to yield a population of primary immune cells resistant to replicative senescence (RRS). In further aspects, the primary immune cells are re-stimulated at least one time, at least two times, at least three time, at least four times, or at least five times. Thus, in particular aspects, provided herein is a method of generating a population of primary immune cells resistant to replicative senescence (RRS), comprising: i) introducing one or more genetic edits into the primary immune cells; ii) culturing the primary immune cells in a culture medium; iii) stimulating the primary immune cells; iv) culturing the primary immune cells in the culture medium; v) re-stimulating the primary immune cells; and vi) culturing the primary immune cells in the culture medium wherein the culturing induces proliferation of the primary immune cells to yield a population of primary immune cells resistant to replicative senescence (RRS). Any suitable stimulus known in the art can be used stimulate the immune cells.

In particular aspects, the primary immune cells undergo at least about a 50-fold expansion, at least about a 500-fold expansion, at least about a 5000-fold expansion, at least about a 250,000-fold expansion, at least about a 500,000-fold expansion, at least about a 10⁶ fold expansion, at least about a 10⁷ fold expansion, at least about a 10⁸ fold expansion, at least about a 10⁹ fold expansion, or at least about a 10¹⁰ fold expansion during culturing. In particular aspects, the population of expanded primary immune cells is resistant to replicative senescence. Furthermore, these cells are not functionally exhausted following long-term expansion and can be directed to carry out cytotoxic function through engagement of their TCRs by a T cell engager antibody or through engagement of a chimeric antigen receptor (CAR), (or through a natural or genetically-introduced TCR).

In particular aspects, the primary immune cells are cultured in a culture medium that includes supportive cytokine(s) but does not include a primary immune cell stimulus. In particular aspects, the primary immune cells undergo expansion during culturing in the absence of feeder cells or stimulation through CD3 and/or their antigen receptor. The ability of the disclosed methods to generate immune cells in the absence of extensive T cell re-stimulation or feeder cells advantageously eliminates the issues of scaling up the methods and producing dysfunctional populations of immune cells.

The methods disclosed herein advantageously provide populations of expanded primary immune cells including human CD8⁺ T cells, human CD4⁺ T cells, human regulatory T cells human gamma-delta T cells, or human natural killer T cells that have the ability to proliferate for substantial periods of time in the absence of re-stimulation through their T cell receptors (TCRs), expanding millions of fold in long-term culture. In particular aspects, the population of primary immune cells are cultured for at least 20 days, at least 30 days, at least 40 days, at least 50 days, at least 60 days, at least 70 days, at least 80 days, at least 90 days, at least 100 days, at least 150 days, at least 200 days, at least 300 days, or at least 400 days.

In further aspects provided herein is an engineered T cell expressing a transgene encoding a B-cell lymphoma-extra large (Bcl-xL) that does not express cyclin-dependent kinase inhibitor 2A (CDKN2A), cyclin-dependent kinase inhibitor 2B (CDKN2B), and/or S-methyl-5′-thioadenosine phosphorylase (MTAP).

In further aspects provided herein is an engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP).

In further aspects provided herein is an engineered T cell expressing a transgene encoding a B-cell lymphoma-extra large (Bcl-XL) that does not express cyclin-dependent kinase inhibitor 2A (CDKN2A), cyclin-dependent kinase inhibitor 2B (CDKN2B), S-methyl-5′-thioadenosine phosphorylase (MTAP) and/or phosphatase and tensin homolog (PTEN).

In certain aspects, the engineered T cell as disclosed herein does not express of one or more endogenous immune related genes in the primary immune cells. In some aspects, the endogenous immune related gene is beta-2 microglobulin (B2M), or T-cell receptor α constant (TRAC).

In certain aspects, the engineered T cell as disclosed herein does not express cluster of differentiation 38 (CD38).

In further aspects the disclosure herein provides an engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), T-cell receptor α constant (TRAC), cluster of differentiation 38 (CD38), and/or phosphatase and tensin homolog (PTEN).

In certain aspects, the engineered T cell as disclosed comprises a polynucleotide that encodes a chimeric antigen receptor (CAR). In some aspects, the CAR binds glypican 3 (GPC3), B-cell maturation antigen (BCMA), or human epidermal growth factor receptor 2 ((HER2); also known as Erb-B2 Receptor Tyrosine Kinase 2 (ERBB2)).

In certain aspects, the engineered T cell as disclosed herein is a CD8+ T cell, a CD4+ T cell, a gamma delta T cell, a mucosal associated invariant T (MAIT) T cell, a natural killer (NK) cell, a natural killer T (NKT) cell, or a combination thereof.

In some aspects, the engineered T cell is resistant to replicative senescence (RRS). In some aspects, the engineered T cell is a CD8⁺ T cell. In some aspects, the engineered T cell is a CD4⁺ T cell. In some aspects, the engineered T cell is human.

Expanded T cell populations disclosed herein are useful for cellular immunotherapies including, without limitation, T cell therapy, adoptive cell therapy (ACT), and CAR T cell therapy.

Expanded populations of T cells populations disclosed herein are useful for treating or preventing various disorders such as a cancer (e.g., a blood malignancy such as lymphoma or leukemia or solid tumors such as melanoma or kidney cancer), autoimmune diseases or an infectious disease such as HIV.

The terms “treatment” or “treat,” as used herein, refer to both therapeutic treatment and prophylactic or preventative measures. Those in need of treatment include subjects having cancer as well as those prone to having cancer or those in cancer is to be prevented. In some aspects, the methods, compositions, and combinations disclosed herein can be used for the treatment of cancer. In other aspects, those in need of treatment include subjects having a tumor as well as those prone to have a tumor or those in which a tumor is to be prevented. In certain aspects, the methods, compositions, and combinations disclosed herein can be used for the treatment of tumors. In other aspects, treatment of a tumor includes inhibiting tumor growth, promoting tumor reduction, or both inhibiting tumor growth and promoting tumor reduction.

In some cases, T cells obtained according to a method provided herein can be administered as a pharmaceutical composition comprising a therapeutically effective amount of T cells as a therapeutic agent (i.e., for therapeutic applications).

The terms “pharmaceutical composition” or “therapeutic composition,” as used herein, refer to a compound or composition capable of inducing a desired therapeutic effect when properly administered to a subject. In some aspects, the disclosure provides a pharmaceutical composition comprising a pharmaceutically acceptable carrier and a therapeutically effective amount of at least one immune cell of the disclosure.

The terms “pharmaceutically acceptable carrier” or “physiologically acceptable carrier,” as used herein, refer to one or more formulation materials suitable for accomplishing or enhancing the delivery of one or more immune cells of the disclosure.

The term “subject” is intended to include human and non-human animals, particularly mammals. In certain aspects, the subject is a human patient.

The terms “administration” or “administering,” as used herein, refer to providing, contacting, and/or delivering a compound or compounds by any appropriate route to achieve the desired effect. Administration may include, but is not limited to, oral, sublingual, parenteral (e.g., intravenous, subcutaneous, intracutaneous, intramuscular, intraarticular, intraarterial, intrasynovial, intrasternal, intrathecal, intralesional, or intracranial injection), transdermal, topical, buccal, rectal, vaginal, nasal, ophthalmic, via inhalation, and implants.

Without limiting the disclosure, a number of aspects of the disclosure are described herein for purpose of illustration.

### EXAMPLES

The Examples that follow are illustrative of specific aspects of the disclosure, and various uses thereof. They are set forth for explanatory purposes only and should not be construed as limiting the scope of the disclosure in any way.

### Example 1: Overexpression of Anti-Apoptotic or Virally-Derived Factors can Provide a Selective Survival Advantage to Transfected T Cells in Long-Term Culture

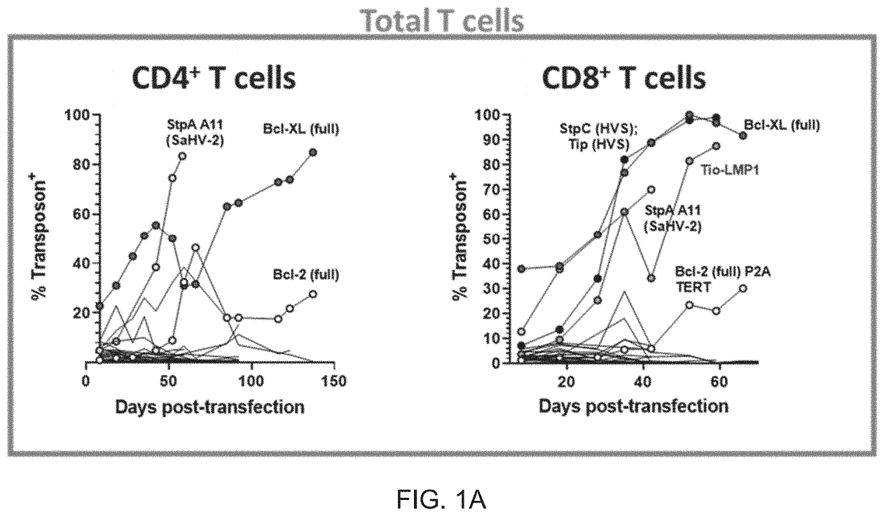

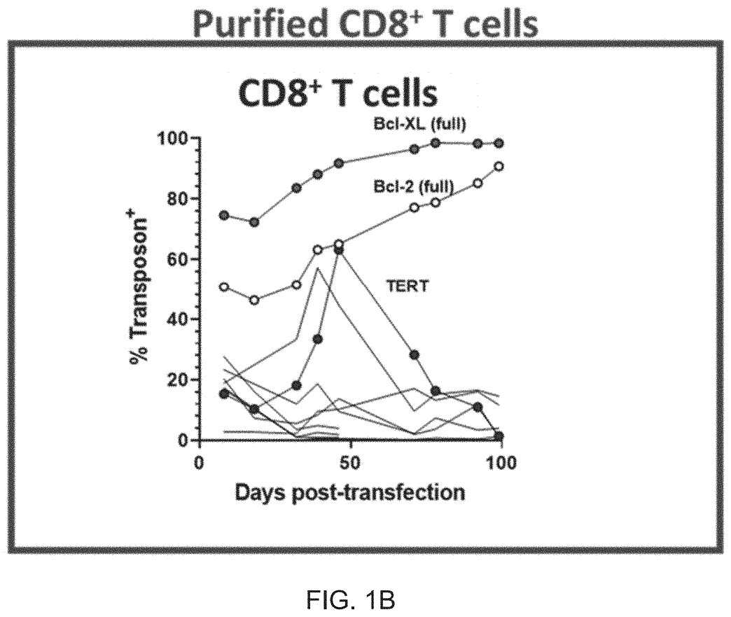

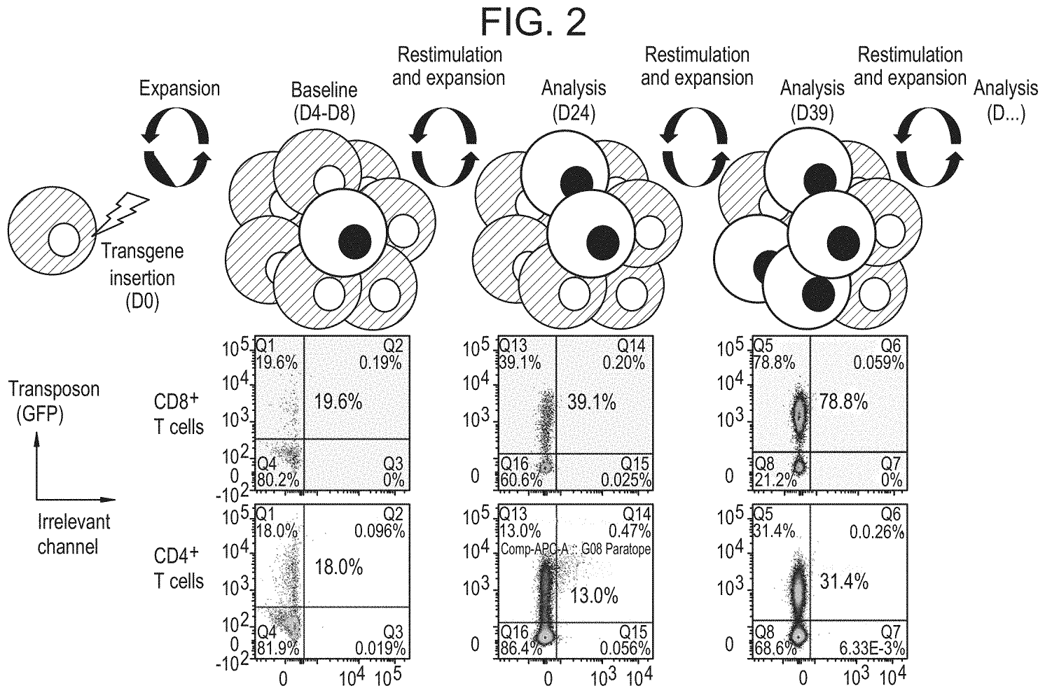

Transposon frequency was assessed in T cell subsets over a period of 66-137 days using flow cytometry. Total primary human T cells were isolated from the blood of healthy donors, activated for 72 hours with Dynabeads (Human T-Activator CD3/CD28) and then transfected with plasmids containing transposons encoding anti-apoptotic factors, virally-derived factors, mutant cytokine receptors, mutant signaling molecules, and/or mutant cell cycle regulatory molecules in addition to a fluorescent reporter. mRNA encoding a transposase was simultaneously transfected into cells to enable chromosomal integration of the transposable elements. In total, 52 transposon constructs were tested across various screens. Four to 8 days after transfection, cells were assessed for baseline transposon incorporation using flow cytometry. Cells were periodically restimulated with Dynabeads to drive them through proliferation and transposon enrichment was assessed using flow cytometry. Molecules that enhance the survival of expanding T cells would be expected to enrich over their starting frequency within the total T cell pool as demonstrated in the CD8⁺ and CD4⁺ T cells. (FIG. 1, FIG. 2). These screens revealed that the anti-apoptotic factor B-cell lymphoma-extra-large (Bcl-xL) consistently enriched in both CD4⁺ and CD8⁺ T cells that were driven through multiple rounds of proliferation over a period of extended in vitro culture, suggesting that this factor may act to enhance the survival of the mature T cells that overexpress it (FIG. 1). Other factors such Bcl-2 and the virally-derived proteins StpA All (Saimiriine gammaherpesvirus 2), StpC and Tip (Herpesvirus saimiri), and a modified Tio-LMP1 (Herpesvirus ateles, Epstein-Barr virus) also demonstrated enrichment in T cell subsets (FIG. 1).

While cells expressing transgenes encoding endogenous anti-apoptotic factors (Bcl-2 and Bcl-xL) or virally-derived factors (StpA All, StpC and Tip, and a modified Tio-LMP1) demonstrated an enhanced ability to survive in long-term culture with repeated stimulation through their TCRs relative to untransfected control cells in the same wells, these cells did not exhibit a large, sustained boost in their proliferative capacities. These data suggest that additional edits may be required to confer the desired phenotype of a T_{REX} cell.

### Example 2: Ablation of Expression of CDKN2A, CDKN2B, and MTAP Substantially Increases the Proliferative Capacity of Primary Human T Cells in Long-Term Culture

Patient-derived leukemic cell lines have been used for years in laboratories to conduct a variety of cellular assays. The transformed nature of these cells can be mapped on to a collection of mutations that are also frequently found in patients with T cell acute lymphoblastic leukemia (T-ALL) (Table 1). Mutations in T-ALL patients can be broken down into several large classes that each presumably contribute to the phenotype and generation of T-ALL cells: gain of activating signals, loss of signal suppressors, loss of cell cycle arrest regulators, as well as modification of pleiotropic factors such as transcription factors, epigenetic regulators, and other cellular machinery.

TABLE 1
Potential T-ALL mutational targets for TREX cell development

| Biological | | | General Strategy for |
|---|---|---|---|
| Function | Pathway or Target | Examples | T_{REX} Cells |
| ACTIVATING | NOTCH | NOTCH1* | AVOID genetic edits; |
| signal | | FBXW7 | significant, broad |
| | | | reaching effects of |
| | | | signaling. |
| | PI3K/mTOR/AKT | AKT*, PI3KCA*, | EDIT and if effective |
| | | mTOR* | express in an inducible |
| | Cytokine | JAK1* JAK3*, | manner to limit |
| | Signaling/Jak/STAT | IL7RA*, STAT5B* | cytokine-independence. |
| | Ras/MAPK | KRAS*, NRAS *, | |
| | | NF1 | |
| | Shared | MYC* | |
| Signal | PI3K/mTOR/AKT | PTEN | EDIT to limit signal |
| SUPPRESSOR | Other | PTPN2 | suppression. |
| Cell Cycle | CDKN2A/2B | CDKN2A/2B | EDIT to limit cell cycle |
| ARREST | RB | RB1 | arrest. |
| Pleiotropic | Translocations | BCL11B, ETV6, | AVOID genetic edits |
| | | GATA3, HOX11*, | due to diverse range of |
| | | HOX11L2*, | biological effects. |
| | | HOXA* LEF1, | |
| | | LMO2*, MYB* | |
| | | MYC* | |
| | | NKX2.1/NKX2.2*, | |
| | | NUP214-ABL1/ | |
| | | ABL1 gain *, | |
| | | RUNX1, TAL1* | |
| | | TLX1*, WT1 | |
| | Epigenetic Modifiers | DNMT3A, EED, | |
| | | EZH2, | |
| | Other | DNM2, CNOT3, | |
| | | RPL5, RPL10, | |
| | | RPL22 | |

*indicates gain of function for example through mutation of the protein, translocations, or mutation of the promoter/enhancer regions as may be the case for a protein such as TERT. Lack of an asterisk indicates loss of function (deletion, loss of function mutations, indels, truncations, etc.).

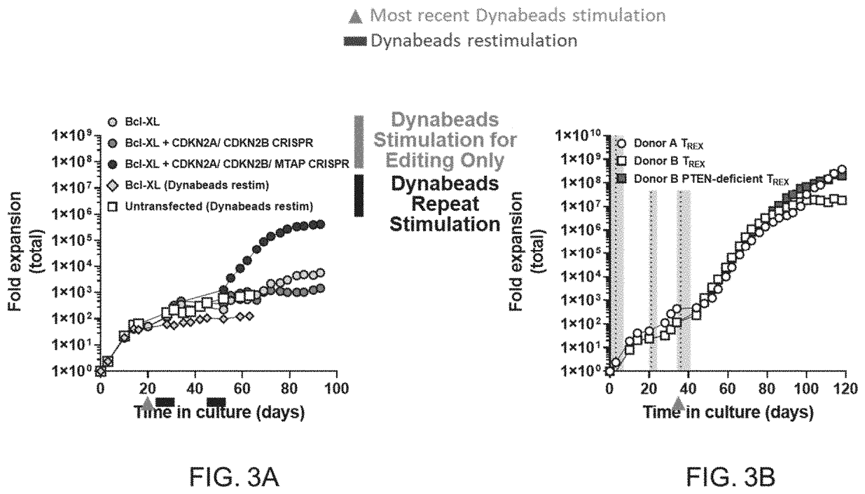

As stated above, it was hypothesized that in addition to providing a survival factor such as Bcl-XL, it may be necessary to modify T cell expression of a collection of the aforementioned genes in order to recreate the desired phenotype (Table 1). Therefore, total CD8⁺ T cells were isolated from the blood of healthy donors, activated using αCD3/αCD28 Dynabeads, and 72 hours later a transgene encoding Bcl-XL was inserted into the collective pool of purified CD8⁺ T cells. These cells were then expanded for a period of 17 days prior to reactivation using αCD3/αCD28 Dynabeads and subsequent ablation of expression of factors identified from leukemic T cell lines and patients with T-ALL. Increased proliferative capacity is one of the primary characteristics that was to be engineered into T_{REX} cells, therefore, effects of ablating expression of molecules from the “Cell Cycle ARREST” bin: cyclin-dependent kinase inhibitor 2A (CDKN2A) and CDKN2B were tested in these cells (FIG. 3A). S-methyl-5′-thioadenosine phosphorylase (MTAP) is chromosomally adjacent to CDKN2A and CDKN2B and is also frequently lost in patients with deletions of CDKN2A and CDKN2B. Accordingly, the effects of ablating expression of MTAP in conjunction with CDKN2A and CDKN2B (FIG. 3A) were tested. These Bcl-XL and CDKN2A/CDKN2B/MTAP-edited cells are subsequently referred to as “T_{REX}+Bcl-xL” cells (Table 2) and the CDKN2A/CDKN2B/MTAP edits (without Bcl-XL) are referred to as “T_{REX}” cells.

TABLE 2
Editing and population terminology

| Term | Specific Edits | Donor |
|---|---|---|
| T_{REX} + Bcl-XL cells | CDKN2A/CDKN2B/MTAP edits + Bcl-xL | 40A30 (“Donor A”) |
| | transgene insertion | and 40B30 (“Donor |
| | | B”) |
| PTEN-deficient | CDKN2A/CDKN2B/MTAP edits + Bcl-xL | 40B32 (“Donor B-2”) |
| T_{REX} cells | transgene insertion + ablation of PTEN | |
| | expression | |

Bcl-xL-edited, Bcl-xL and CDKN2A/CDKN2B-edited, and T_{REX}+Bcl-xL cells were maintained in culture for nearly 100 days without additional stimulation through their TCRs and total fold expansion of each population was assessed (FIG. 3A). Proliferation of T_{REX}+Bcl-xL cells diverged from the other groups approximately 31 days after introducing these edits, and T_{REX}+Bcl-xL cell expansion was sustained in the absence of additional TCR stimulation, achieving a more than 400,000-fold expansion during this period. In contrast, Bcl-xL-edited and Bcl-xL/CDKN2A/CDKN2B-edited CD8⁺ T cells achieved 73-286-fold lower levels of expansion in this time (FIG. 3A, Table 3). Furthermore, even when unedited or Bcl-xL-edited cells were repeatedly restimulated through their TCRs using αCD3/αCD28 Dynabeads to drive proliferation, they achieved only low levels of total fold expansion (FIG. 1A, Table 3), well below those of the T_{REX}+Bcl-xL cells.

TABLE 3
CD8⁺ T cell proliferation

| | Total Fold | Total Fold |
|---|---|---|
| | Expansion | Expansion |
| Population | (Day 59) | (Day 93) |
| Bcl-XL cells | 554 | 5,677 |
| Bcl-XL + CDKN2A/CDKN2B | 926 | 1,459 |
| CRISPR cells | | |
| Bcl-XL + | 8,306 | 417,948 |
| CDKN2A/CDKN2B/MTAP | | |
| CRISPR (T_{REX} + Bcl-XL) cells | | |
| Bcl-XL cells (Dynabeads restim) | 121 | N/A |
| Untransfected cells (Dynabeads | 689 | N/A |
| restim) | | |

While T_{REX}+Bcl-xL cells demonstrated a substantially enhanced proliferative capacity relative to control CD8⁺ T cells from the same donor, further experiments were conducted to test whether these edits could confer a similar phenotype in other donors and whether it was possible to further enhance the T_{REX}+Bcl-xL phenotype by ablating expression of signal suppressors that are frequently mutated in patients with T-ALL (Table 1). The phosphatase and tensin homolog (PTEN) locus demonstrates frequent loss-of-function mutations in patient-derived leukemic cell lines and patients with T-ALL and is known to negatively regulate cell cycle progression (Table 1). T_{REX}+Bcl-xL cells were generated as above from two different donors (40A30 and 40B30) and expression of PTEN was ablated in one of these T_{REX}+Bcl-xL lines (40B32) approximately 2 weeks after “triplex” editing (FIG. 3B). Both sets of healthy donor-derived T_{REX} cells (lacking CDKN2A/CDKN2B/MTAP) exhibited substantial proliferative capacity in the absence of additional TCR stimulation, achieving >3.7e8 and >1.8e7 total fold expansion by day 118 in culture. Furthermore, in agreement with its established role in negatively regulating cell cycle progression through control of AKT signaling, ablation of PTEN expression in T_{REX}+Bcl-xL cells further enhanced the proliferative capacity of Donor B-2 T_{REX}+Bcl-xL cells, allowing these cells to reach >2.0e8 total fold expansion by day 118 in culture (FIG. 3B). The additive effects of ablation of PTEN expression took 49 days to emerge, reflecting low initial editing efficiency or a late competitive advantage of this edit after the cells have expanded >3e6 fold.

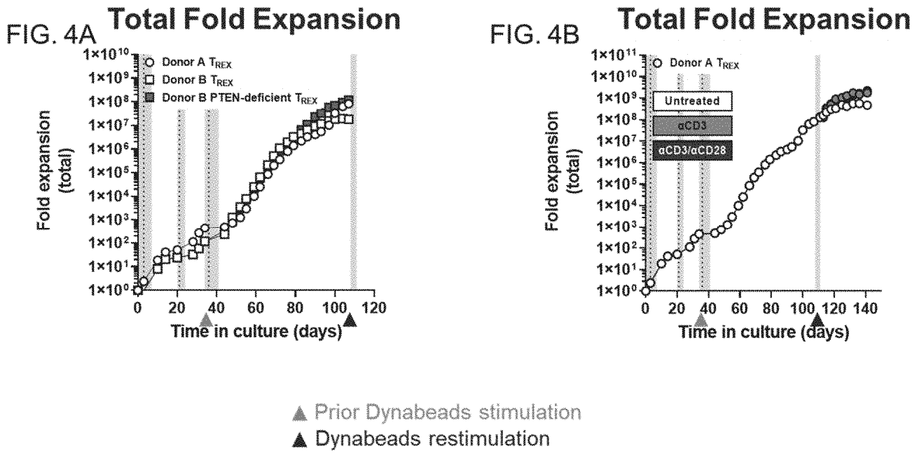

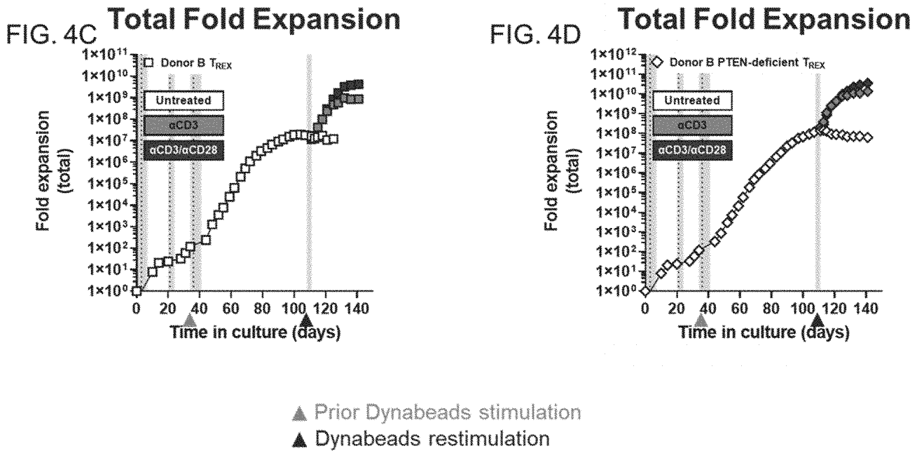

While T_{REX}+Bcl-xL cells with intact PTEN expression expanded dramatically in the absence of additional TCR stimulation, their proliferation ultimately slowed relative to T_{REX}+Bcl-xL cells deficient in PTEN expression (FIG. 3B). Furthermore, even PTEN-deficient T_{REX}+Bcl-xL cells eventually demonstrated decreased rates of proliferation (FIG. 3B). In order to determine if restimulation of T_{REX}+Bcl-xL cells in the presence or absence of co-stimulation could serve as a viable alternative or complementary approach to ablation of PTEN expression, it was tested whether αCD3 or αCD3/αCD28 Dynabeads could jumpstart cells back into cell cycle (FIG. 4). T_{REX}+Bcl-xL or PTEN-deficient T_{REX}+Bcl-xL cells were left untreated or restimulated with Dynabeads as above, debeaded, and total fold expansion of each population was tracked (FIG. 4). Restimulation substantially enhanced the ability of T_{REX}+Bcl-xL and PTEN-deficient T_{REX}+Bcl-xL lines to expand.

### Example 3: T_{REX}+Bcl-xL Cells Resemble Primary Human T Cells in Terms of Cytokine Dependence and Cell Phenotype

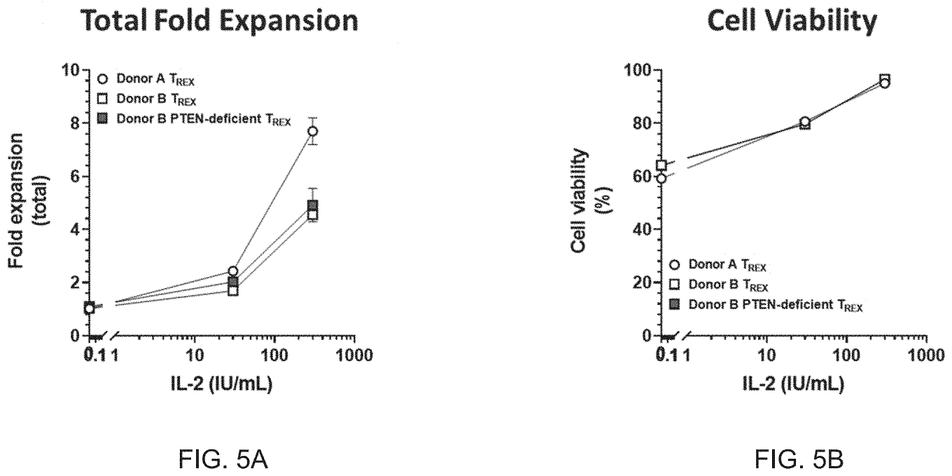

Primary T cells are dependent on cytokines such as IL-2 for survival and proliferation in vitro and in vivo, however, some leukemic cell lines grow independently of IL-2. T_{REX}+Bcl-xL cells and PTEN-deficient T_{REX}+Bcl-xL cells were generated in media containing IL-2. It was investigated whether these cells still resemble normal primary human T cells in regards to cytokine dependence by tracking cell proliferation and survival across a range of IL-2 concentrations over a period of 6 days in culture (FIG. 5). In line with normal T cells, T_{REX}+Bcl-xL cells and PTEN-deficient T_{REX}+Bcl-xL cells were highly dependent on IL-2 for both proliferation and survival.

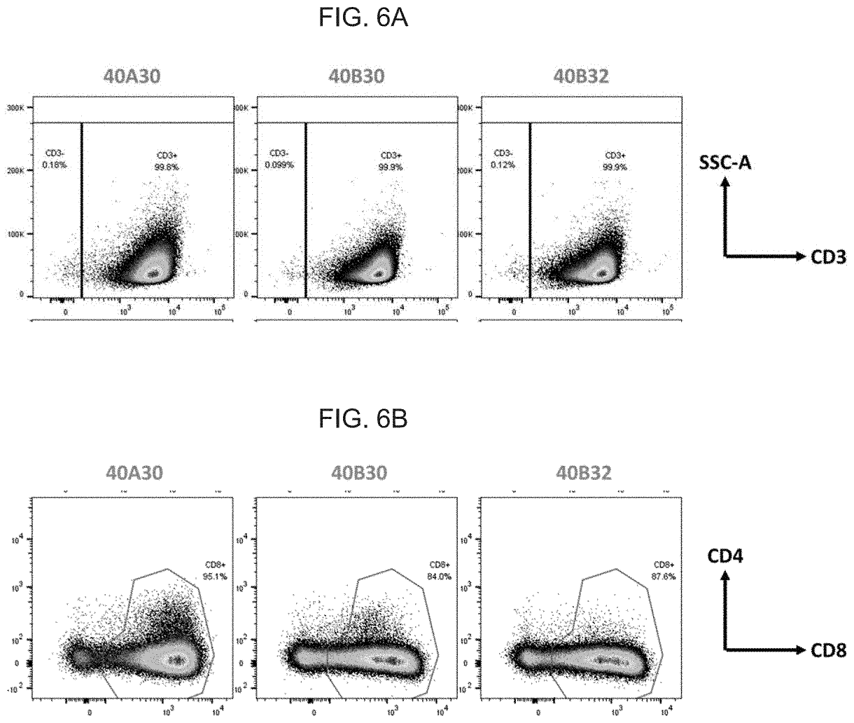

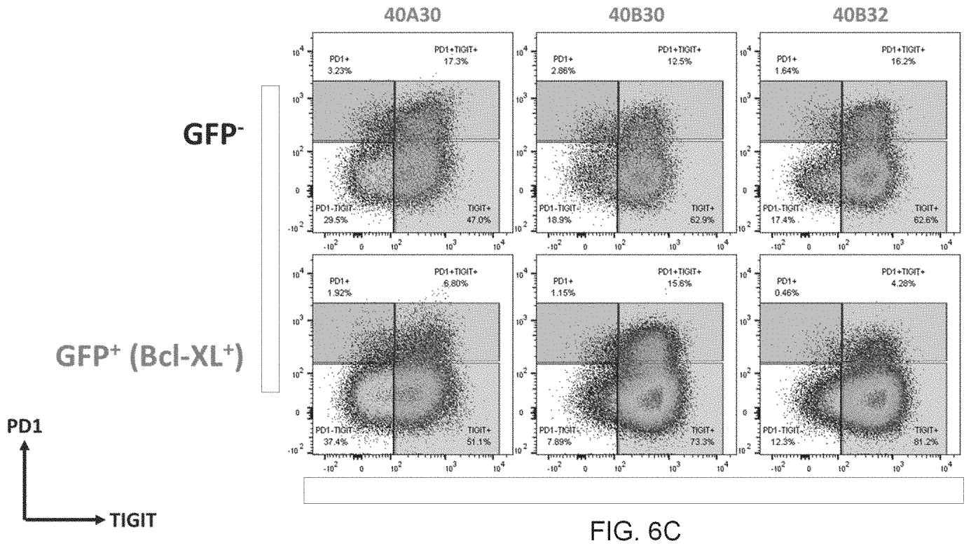

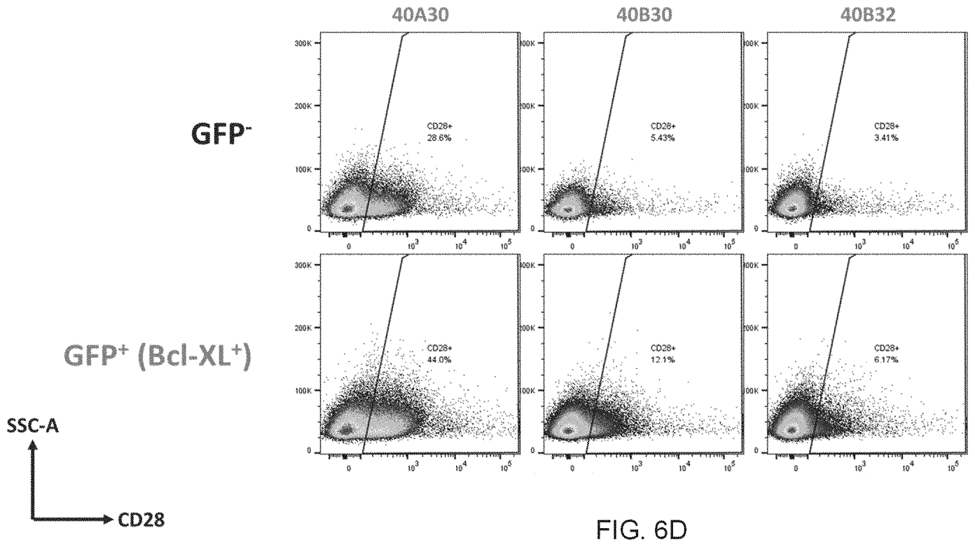

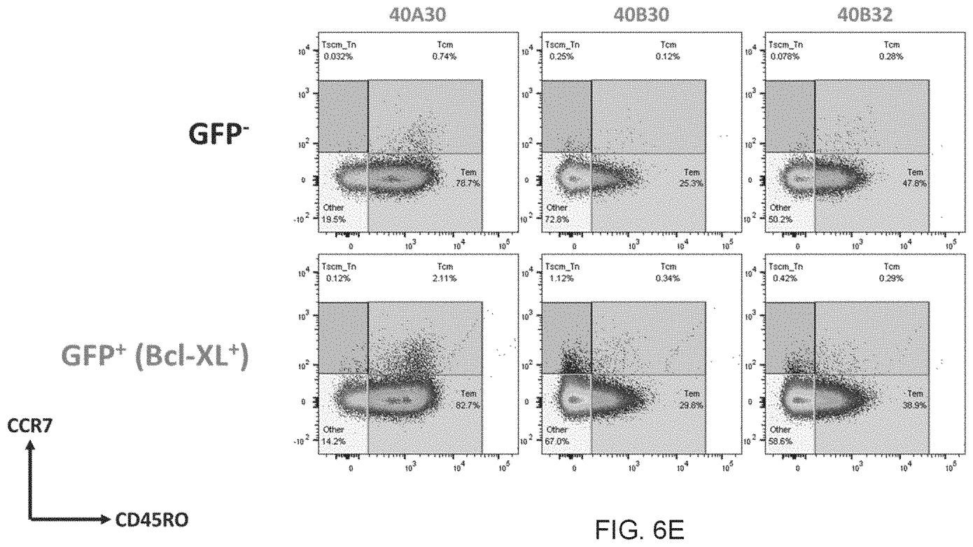

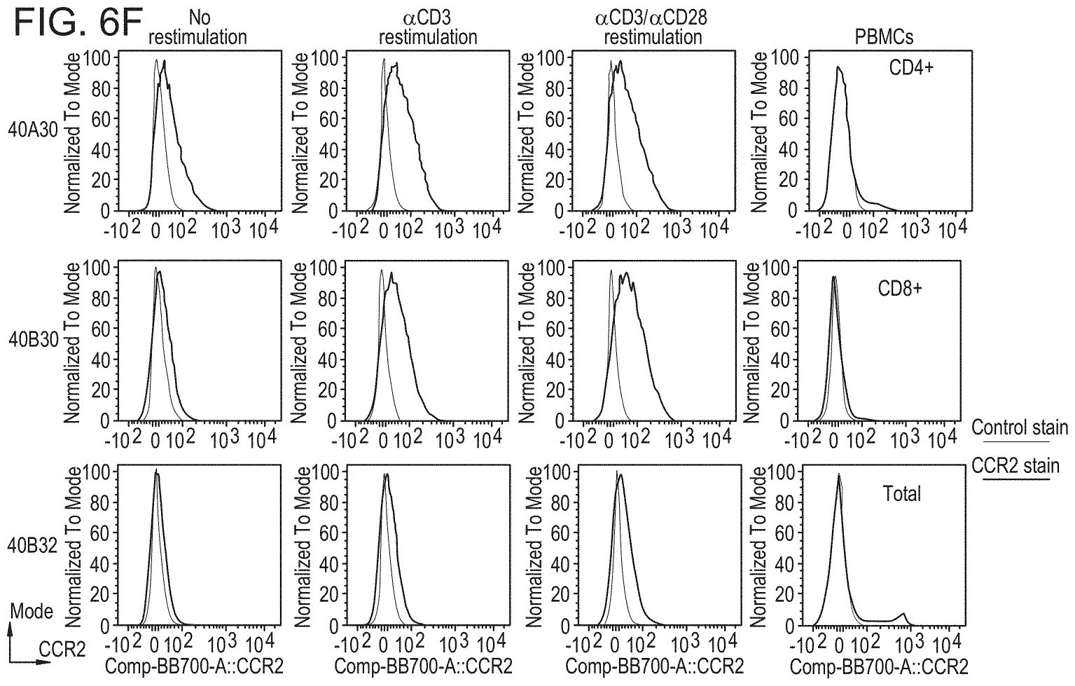

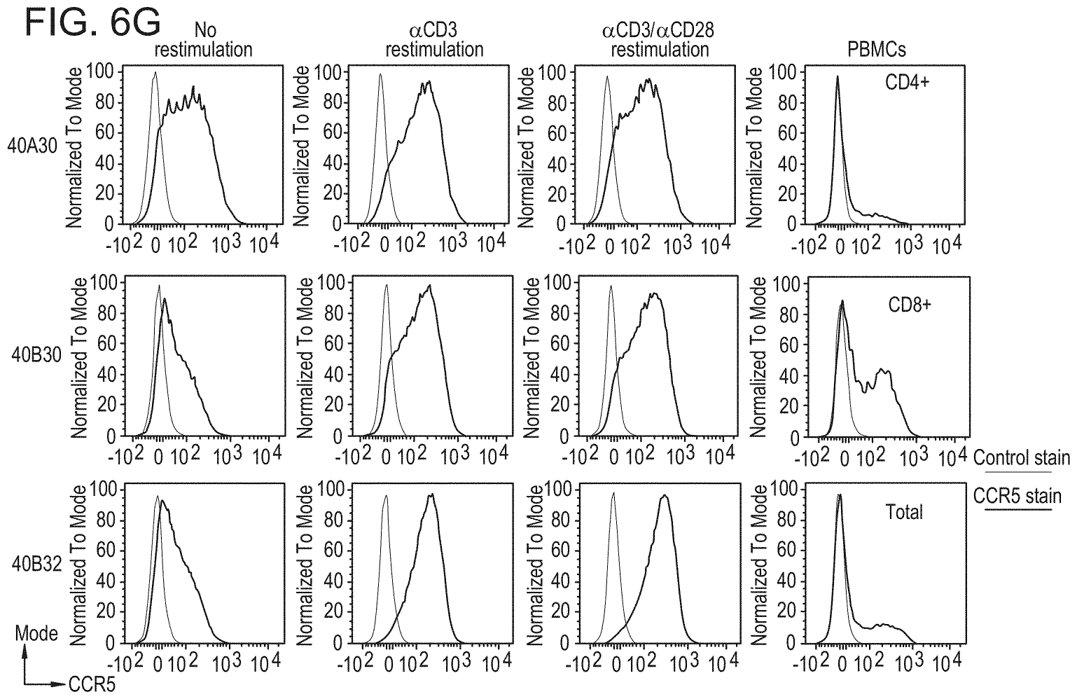

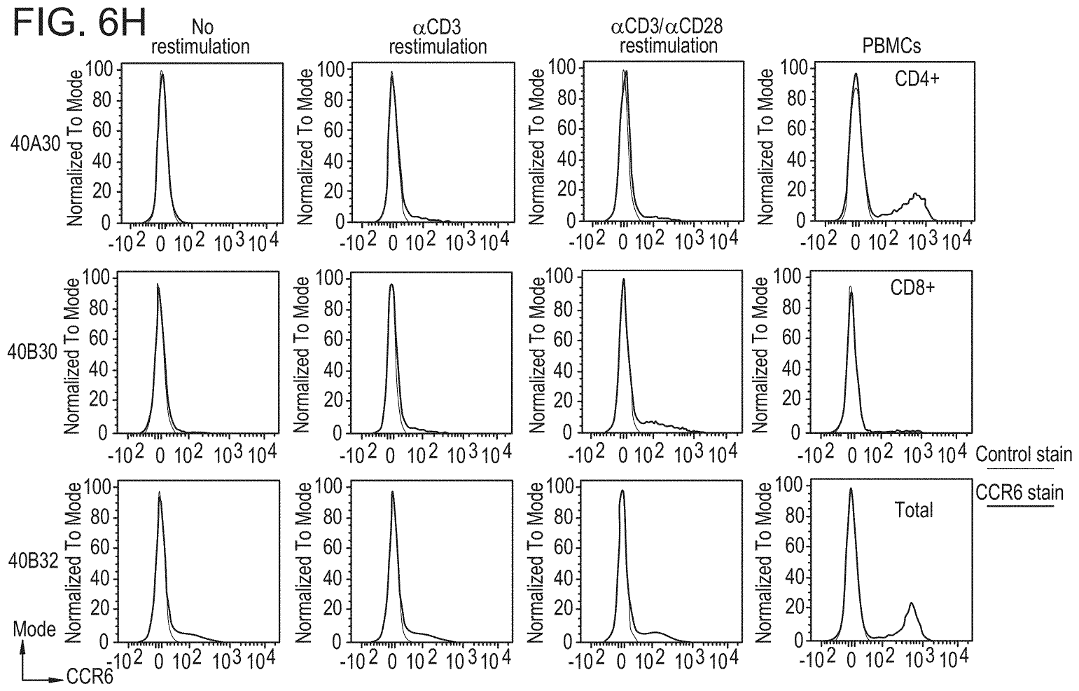

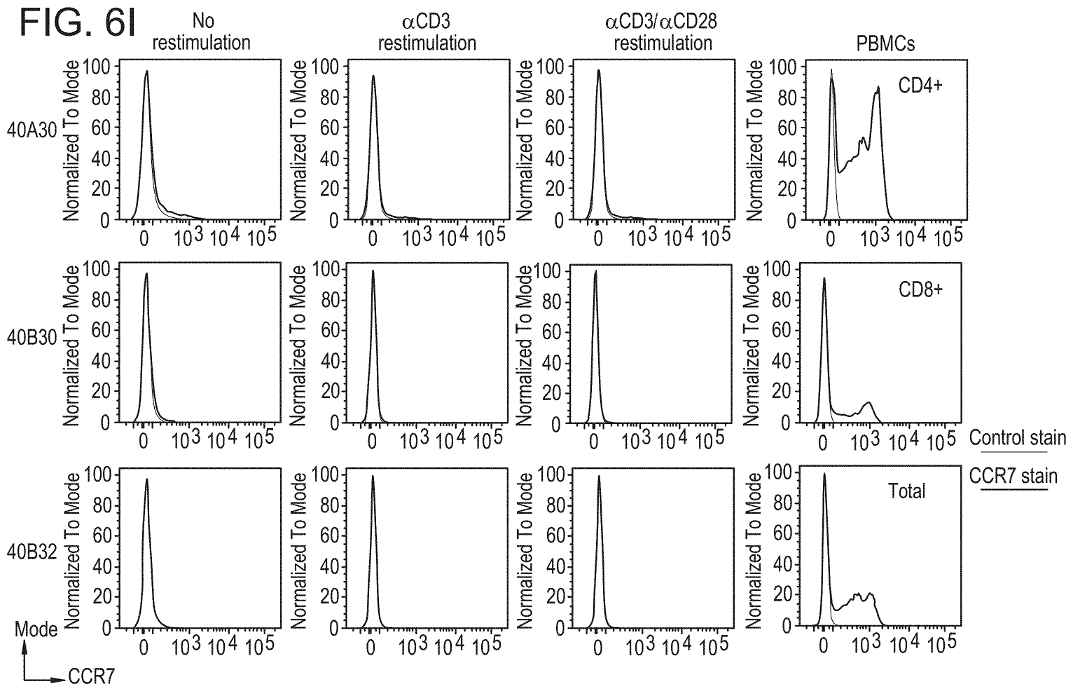

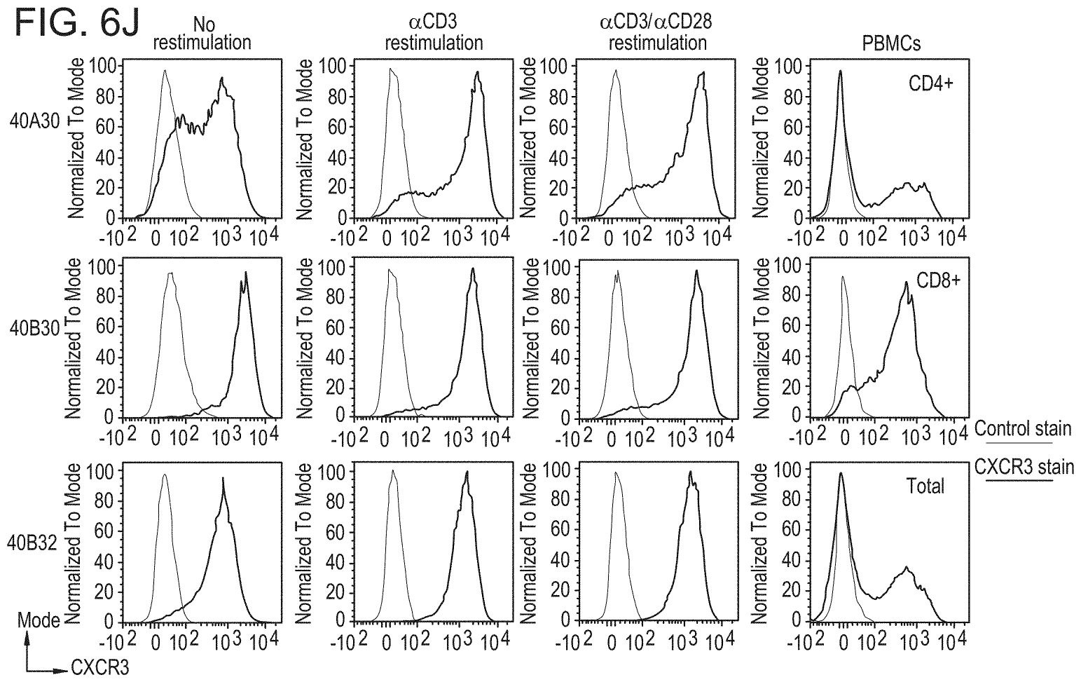

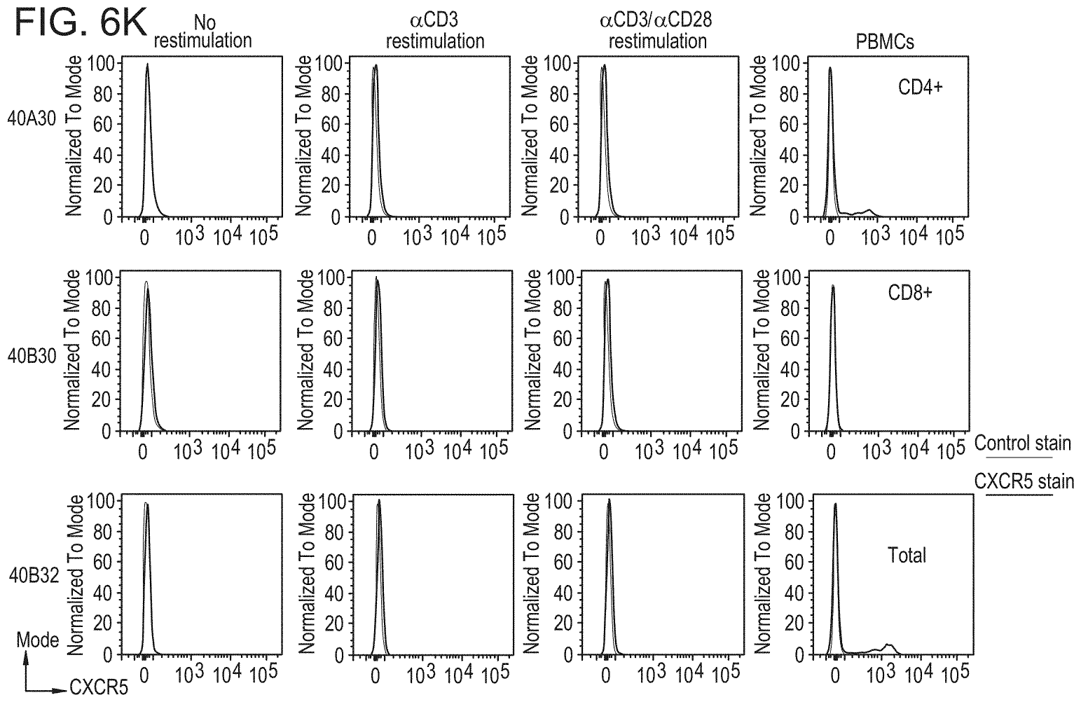

It was also tested whether T_{REX}+Bcl-xL cells and PTEN-deficient T_{REX}+Bcl-xL cells maintain phenotypes similar to normal T cells after modification and extended in vitro culture or whether these conditions drive T_{REX}+Bcl-xL cells to an exhausted phenotype (FIG. 6). All three T_{REX}+Bcl-xL lines maintained expression of cell surface CD3 and CD8 (FIGS. 6A and 6B). Furthermore, they expressed variable levels of activation markers such as PD1 and TIGIT (FIG. 6C) and maintained expression of CD28 in a donor-dependent manner (FIG. 6D). Finally, these T_{REX}+Bcl-xL lines exhibited differentiation phenotypes defined by surface expression of CD45RO and CCR7 that tracked in a donor-dependent manner (FIG. 6E). These data suggest that despite substantial proliferation and an extended duration of in vitro culture, T_{REX}+Bcl-xL cells resemble normal T cells and do not exhibit a surface phenotype associated with a dysfunctional state.

Chemokine receptors are important for trafficking of immune cells to sites of inflammation. Therefore, T_{REX}+Bcl-xL cells, PTEN-deficient T_{REX}+Bcl-xL cells, restimulated T_{REX}+Bcl-xL cells, and restimulated PTEN-deficient T_{REX}+Bcl-xL cells were analyzed for expression of the chemokine receptors CCR2, CCR5, CCR6, CCR7, CXCR3, and CXCR5 using flow cytometry (FIG. 6F-K). T_{REX}+Bcl-xL lines and PTEN-deficient T_{REX}+Bcl-xL lines demonstrated expression of CCR2 (FIG. 6F), CCR5 (FIG. 6G), and CXCR3 (FIG. 6J). Expression of CCR6 (FIG. 6H) was heterogenous, while expression of CCR7 (FIG. 6I) and CXCR5 (FIG. 6K) was low to absent. Therefore, T_{REX}+Bcl-xL cells and PTEN-deficient T_{REX}+Bcl-xL cells maintain expression of key chemokine receptors that will enable them to traffic to sites of inflammation.

### Example 4: T_{REX}+Bcl-xL Cells are Cytotoxic

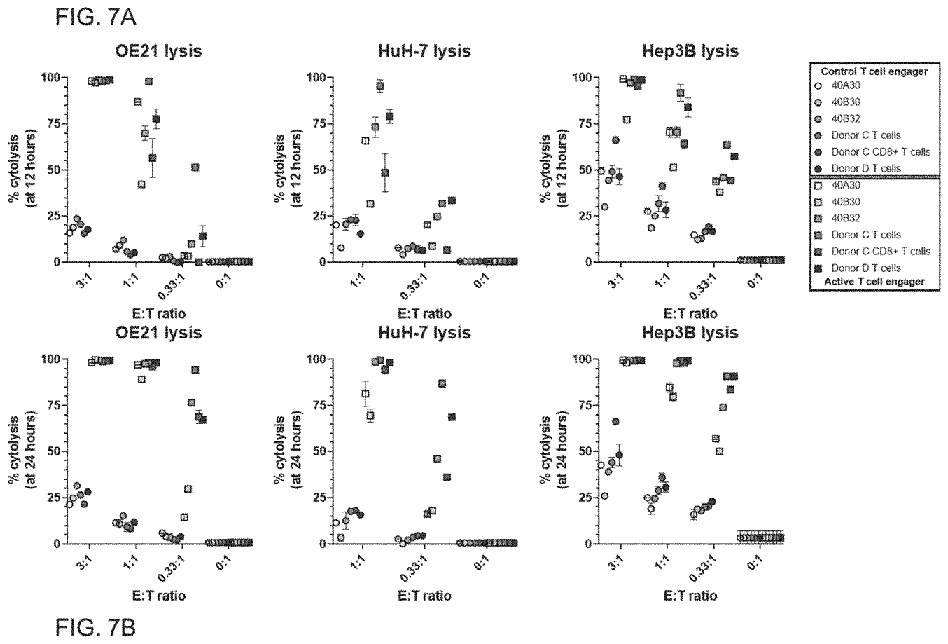

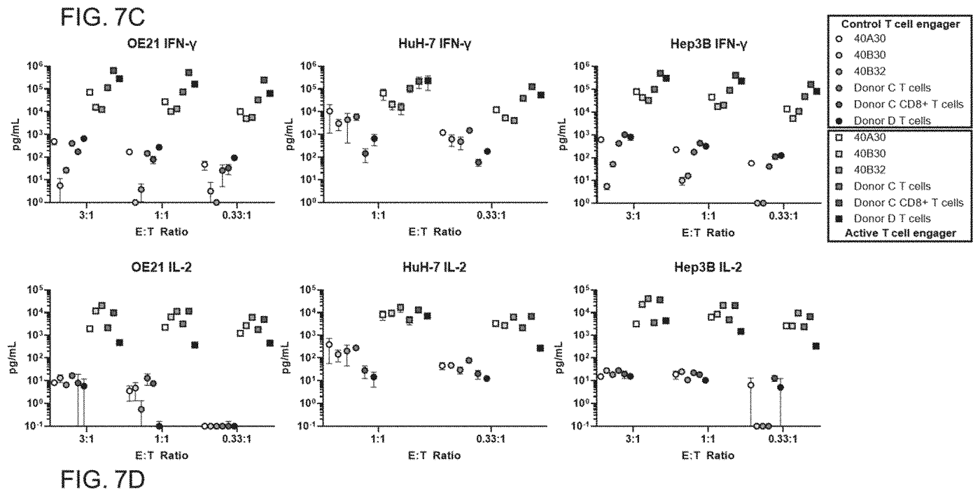

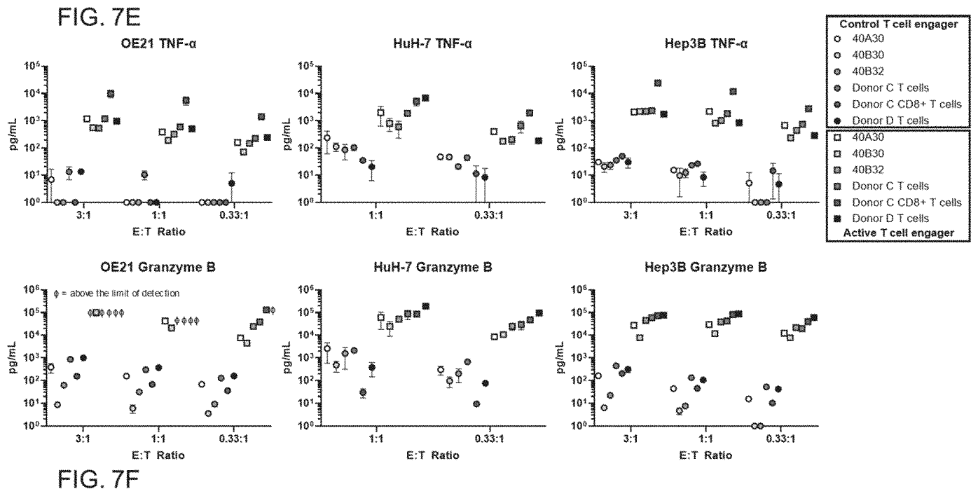

Having established that T_{REX}+Bcl-xL lines resemble normal primary human T cells, it was determined whether T_{REX}+Bcl-xL cells maintain potent cytotoxic function after long-term culture and expansion. A T cell engager was used in the presence of target tumor cells and the impedance-based xCELLigence platform to quantify T_{REX}+Bcl-xL cell cytotoxic function (FIG. 7). Day 80 T_{REX}+Bcl-xL lines demonstrated a comparable ability to lyse target tumor cells in the presence of the T cell engager as unmodified primary total T cells and unmodified primary CD8⁺ T cells (FIGS. 7A and 7B). Supernatants from these co-cultures were collected 72 hours after the addition of effector cells and the active T cell engager or control T cell engager molecule and analyzed for the presence of interferon γ (IFN-γ), IL-2, tumor necrosis factor α (TNF-α), and granzyme B (FIG. 7C-7F). T_{REX}+Bcl-xL lines produced lower levels of these cytokines relative to unmodified primary T cells despite a similar capacity to lyse target cells in an antigen-dependent manner. These data indicate that even after 80 days in culture and substantial expansion, T_{REX}+Bcl-xL cells are not functionally exhausted and maintain their cytotoxic potential.

### Example 5: T_{REX}+Bcl-xL Cells can Produce Functional CAR-T_{REX} Cells

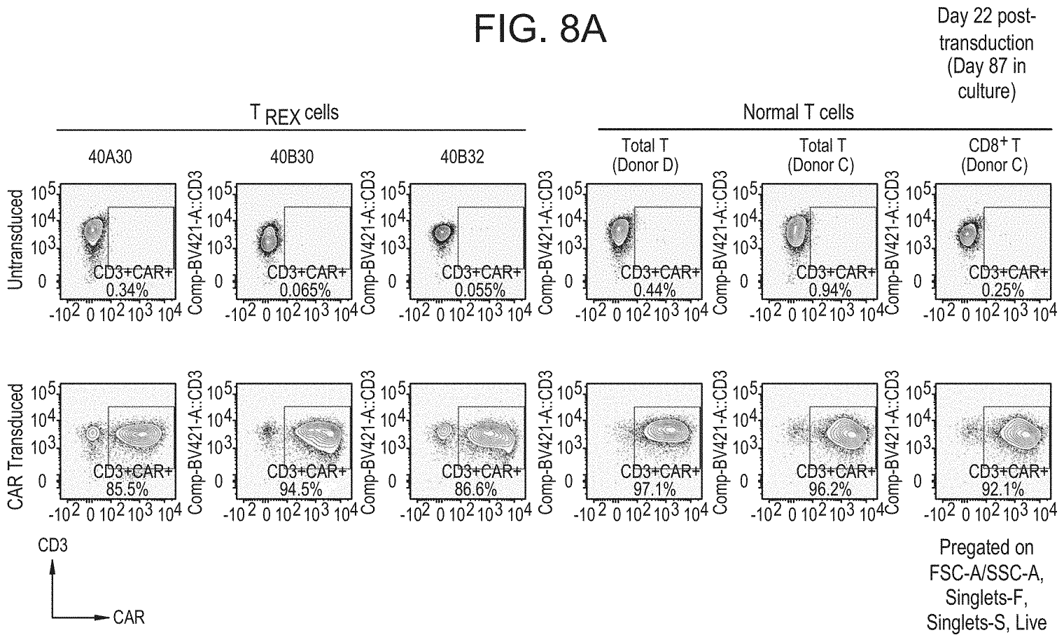

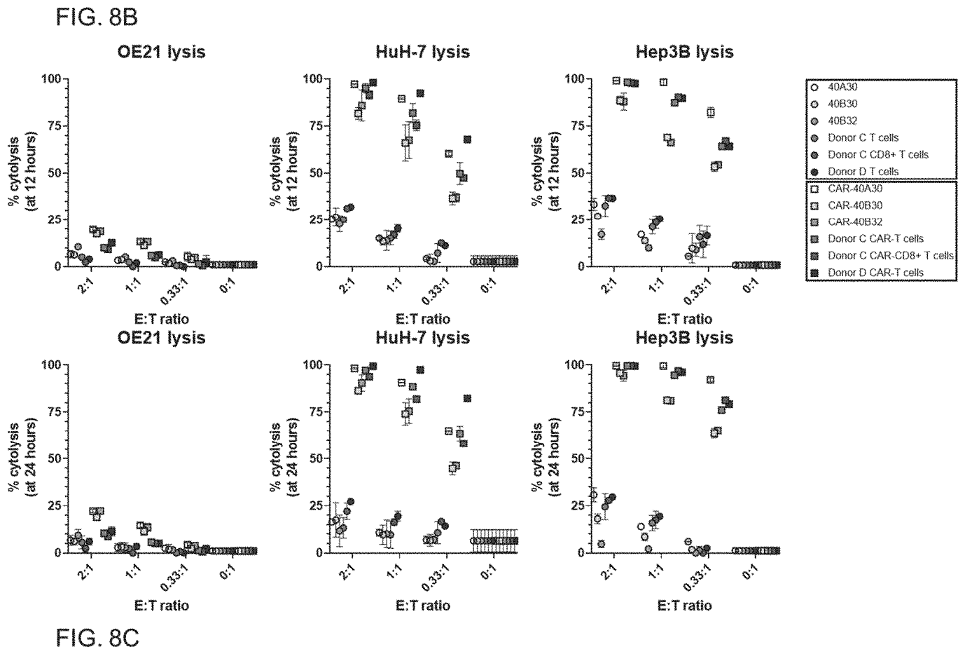

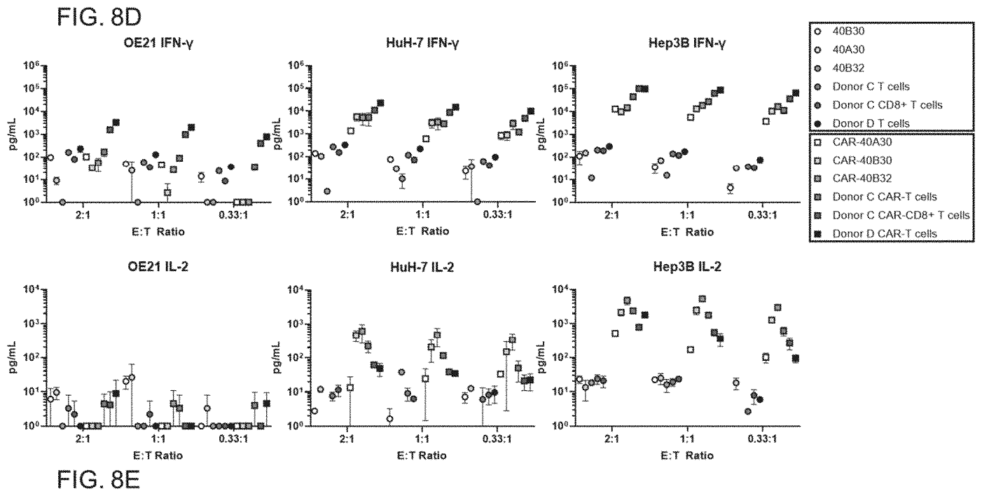

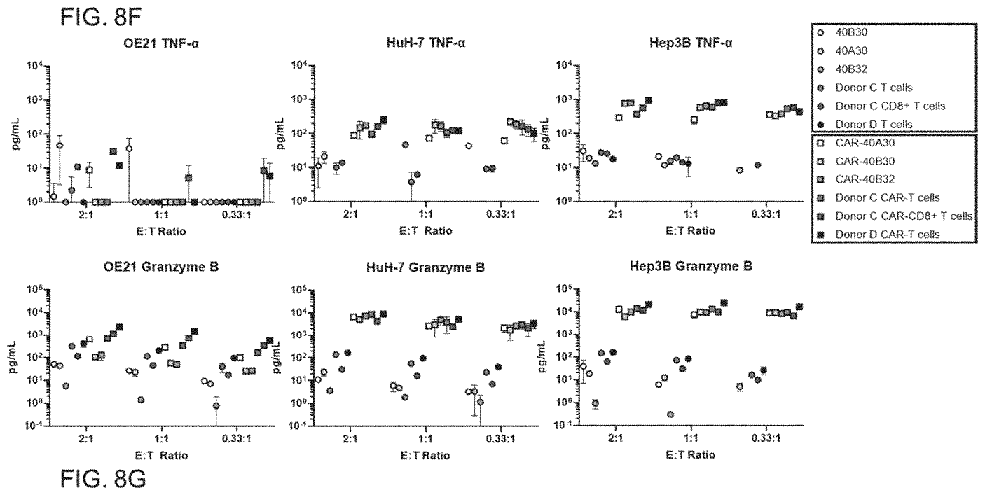

In order to develop T_{REX}+Bcl-xL cells into a potential cellular therapy these cells must be capable of expressing a targeting molecule such as a chimeric antigen receptor (CAR) to direct their cytotoxic function. The three T_{REX}+Bcl-xL cell lines generated as described above were transduced with a lentivirus encoding a CAR that recognizes glypican 3 (GPC3). Surface expression of the GPC3 CAR was subsequently measured using flow cytometry (FIG. 8A). Each T_{REX}+Bcl-xL line was found to successfully express the GPC3 CAR at levels similar to normal primary total T cells and normal primary CD8⁺ T cells (FIG. 8A).

CAR-directed cytotoxic function of T_{REX}+Bcl-xL cells was assessed by performing impedance-based xCELLigence assays using target tumor cells with varying degrees of antigen expression: OE21 (antigen-negative), HuH-7 (antigen-intermediate), and Hep3B (antigen-high) (FIGS. 8B and 8C). T_{REX}+Bcl-xL cells rapidly lysed target tumor cells in a CAR- and antigen-specific manner at levels similar to normal CAR-T cells and normal CAR-CD8⁺ T cells (FIGS. 8B and 8C). Supernatants were harvested from these co-cultures 72 hours after T cell addition and subsequently analyzed for secretion of IFN-γ, IL-2, TNF-α, and granzyme B (FIG. 8D-8G). In agreement with their potent cytotoxic function, CAR-T_{REX}+Bcl-xL cells demonstrated a comparable ability to secrete effector cytokines as normal CAR-T cells and normal CAR-CD8+ T cells (FIG. 8D-8G). However, in general, IFN-γ and TNF-α levels were found to be lower in CAR-T_{REX}+Bcl-xL cells.

These data confirm that T_{REX}+Bcl-xL and PTEN-deficient T_{REX}+Bcl-xL cells are capable of expressing a CAR and carrying out CAR-directed cytotoxic function in an antigen-dependent manner even after significant in vitro expansion.

### Example 6: T_{REX} Cells Traffic to Similar Locations as Primary CD8⁺ T Cells and are Responsive to IL-2 In Vivo

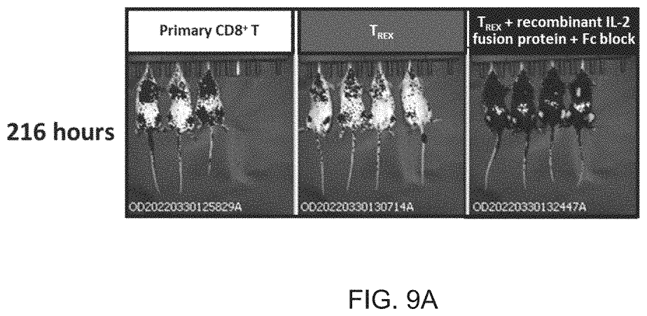

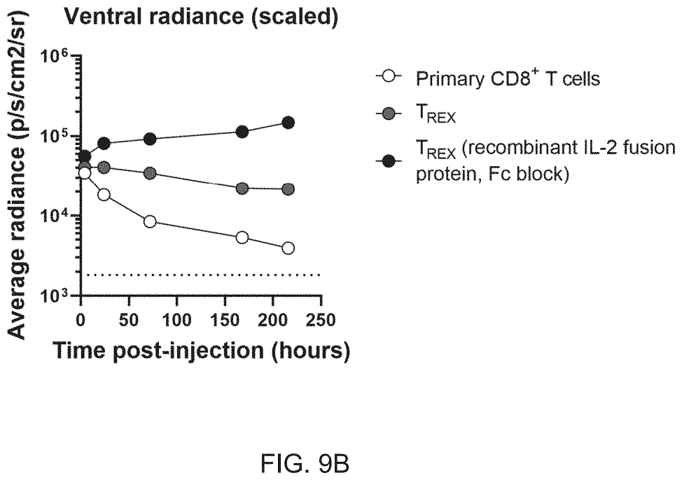

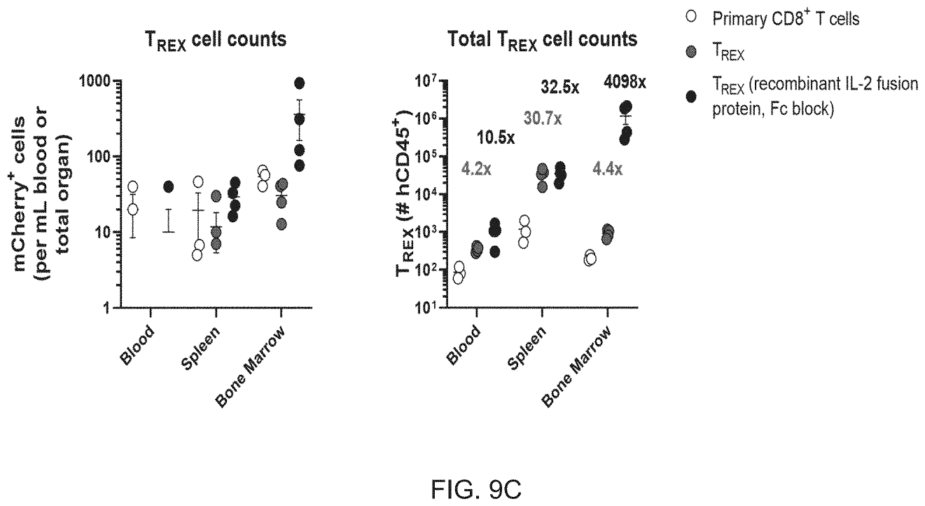

Cytokine cues can be used to modulate activity and expansion of human and murine T cells (Zhang et al., Science Translational Medicine, 22 Dec. 2021, Vol 13, Issue 625; Aspuria et al., Science Translational Medicine, 22 Dec. 2021, Vol 13, Issue 625) therefore T_{REX} cells were assessed for their capacity to respond to different human cytokines in vivo. Briefly, primary human CD8⁺ T cells or 278-day old T_{REX} cells were labeled with a luciferase reporter and 3E6 luciferase-expressing cells were infused into NSG mice with or without supplementation with a recombinant human IL-2 fusion protein. Mice were imaged using an IVIS Optical Imaging system to detect luciferase-expressing T cells (FIGS. 9A and 9B). As shown in FIG. 9A, imaging at 216 hours indicated similar localization of primary human CD8⁺ T cells and T_{REX} cells in mice. Further, mice supplemented with a recombinant human IL-2 fusion protein demonstrated enhanced proliferation of T_{REX} cells (FIG. 9A, right). Ventral radiance was graphed over time (FIG. 9B) and similarly demonstrates the ability of T_{REX} cells to respond to exogenously supplemented IL-2 in vivo. Mice were sacrificed 10 days after adoptive cell transfer and their blood, spleens, and bone marrow were assessed for the presence of primary CD8⁺ T cells or T_{REX} cells (FIG. 9C). While T_{REX} cells were found in similar organs as primary CD8⁺ T cells (FIG. 9C), they demonstrated slower decay kinetics and administration of a recombinant human IL-2 fusion protein could further enhance T_{REX} cell numbers in the blood and bone marrow of treated mice. These data suggest that T_{REX} cells home to similar sites as primary CD8⁺ T cells and maintain responsiveness to exogenous cytokine cues.

### Example 7: CAR-T_{REX} Cells Respond to IL-2 and IL-15 In Vivo

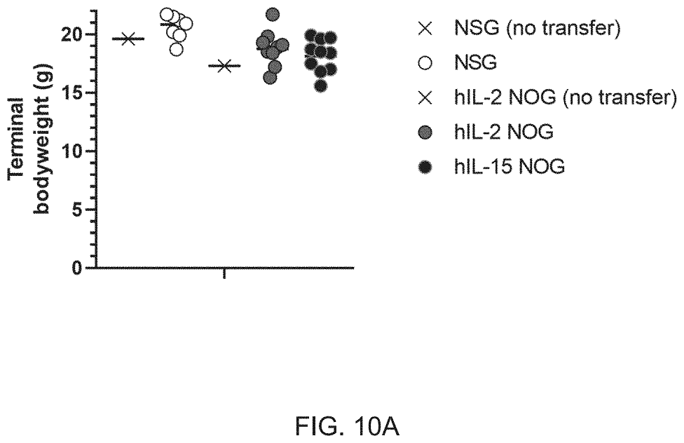

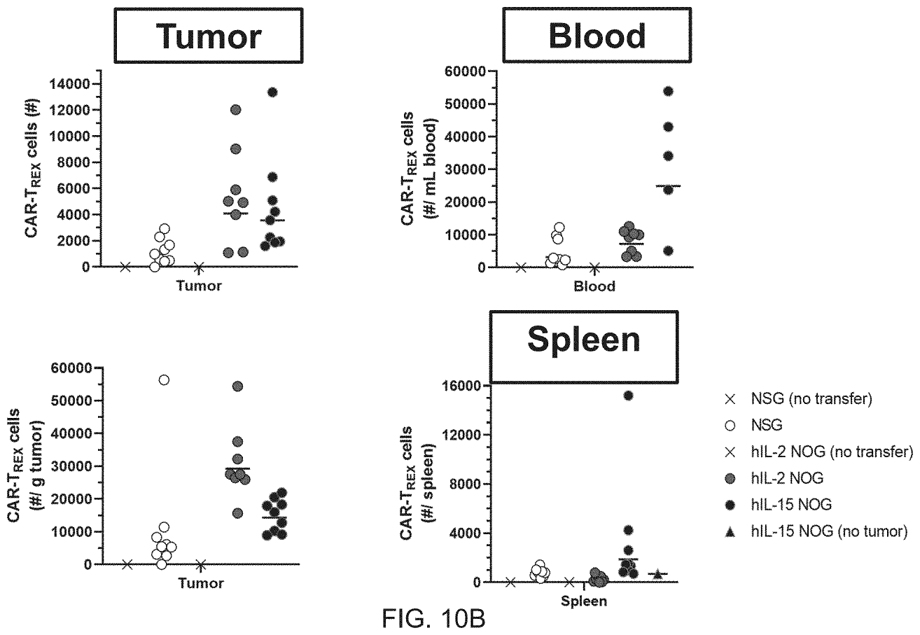

GPC3 targeting CAR-T_{REX}+Bcl-xL cells were assessed for their capacity to respond to different human cytokines in vivo. NSG, hIL-2 NOG, or hIL-15 NOG mice were inoculated with GPC3 expressing Hep3B tumor cells. Once tumors were established, mice were left untreated or were treated with 10E6 GPC3 targeting CAR-T_{REX}+Bcl-xL cells. CAR-T_{REX}+Bcl-xL cells were 121 days at the time of infusion. Mice were sacrificed 8 days after CAR-T_{REX}+Bcl-xL cell infusion and bodyweights were measured (FIG. 10A) with no discernable differences observed indicating lack of toxicity. Tumors, blood, and spleens were harvested and analyzed for the presence of CAR-T_{REX}+Bcl-xL cells (FIG. 10B). CAR-T_{REX}+Bcl-xL cell numbers were enhanced in tumor-bearing hIL-2 NOG and hIL-15 NOG mice indicating that CAR-T_{REX}+Bcl-xL cells are capable of responding to exogenous cytokine cues in vivo. Expansion profiles were specific to the particular cytokine support that was provided (FIG. 10B).

### Example 8: CAR-T_{REX} Cells Target Solid Tumors In Vivo

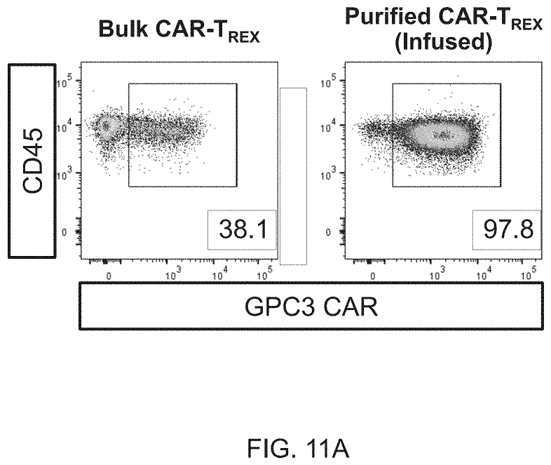

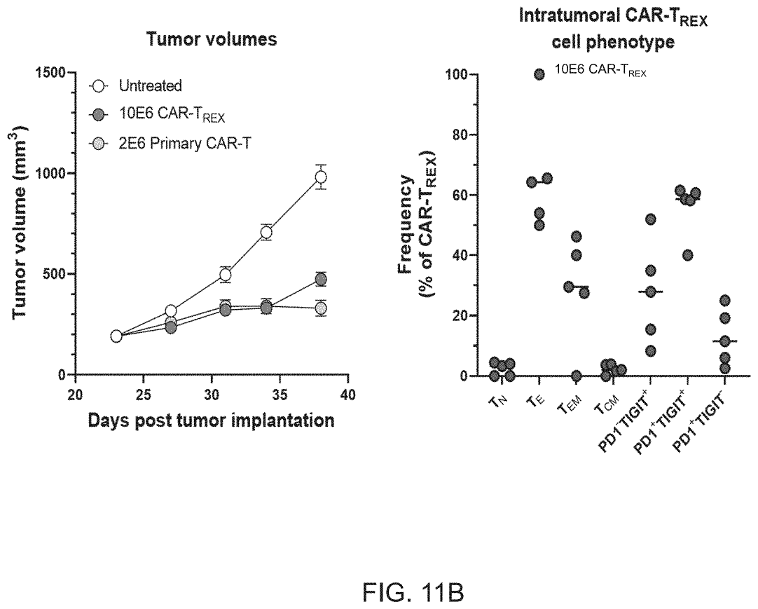

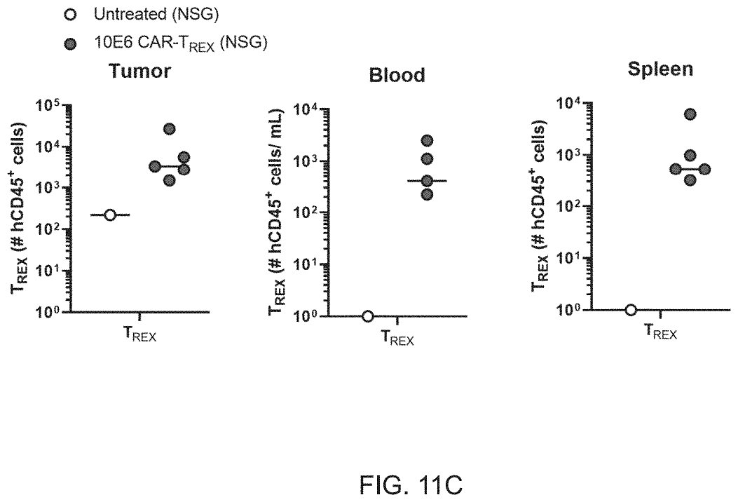

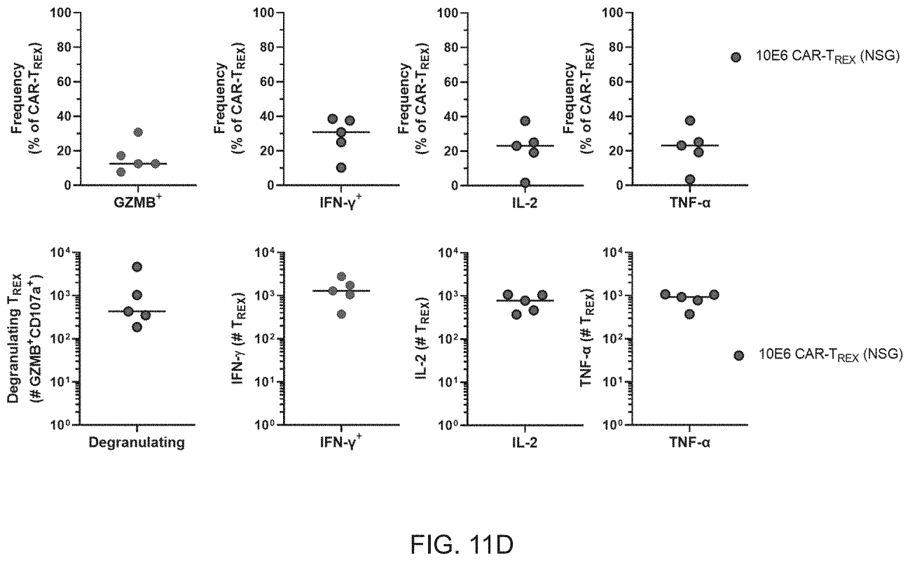

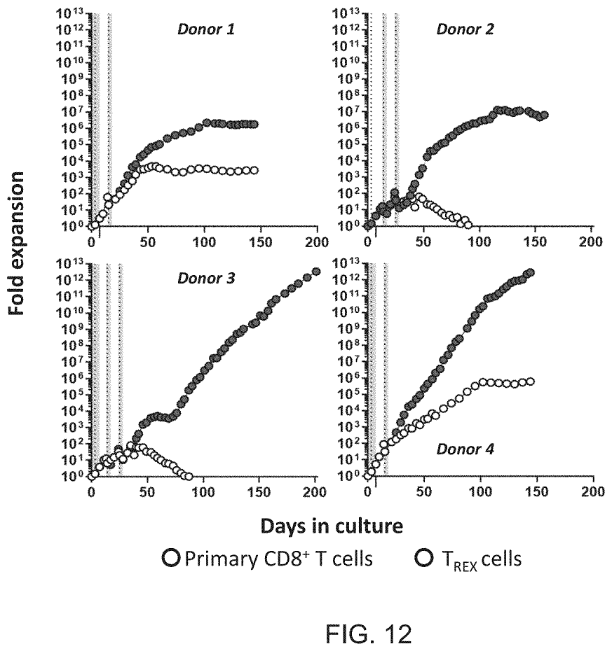

The capacity of GPC3 targeting CAR-T_{REX} cells to control solid tumors was determined. Hep3B tumors were established in NSG mice and then 92 day old purified CAR-T_{REX} cells (FIG. 11A) were infused into mice. 10E6 CAR-T_{REX} cells or 2E6 CAR-T cells were infused and tumor volumes were then measured and graphed over time (FIG. 11B, left). CAR-TREX cells exhibited tumor growth inhibition and control of Hep3B tumors. 18 days post-CAR-TREX cell transfer, mice were sacrificed and tumors, blood, and spleen were harvested for further analysis (FIGS. 11B, 11C, and 11D). Intratumor CAR-T_{REX} cell phenotypes were examined (FIG. 11B), and CAR-T_{REX} cell numbers were determined in these different tissues (FIG. 11C). CAR-T_{REX} cells were found in highest numbers in the tumors of mice and these cells demonstrated an activated phenotype where they were actively secreting effector cytokines and degranulating (FIG. 11D). T_{REX} cells additionally demonstrated an enhanced capacity to proliferate in vitro and can be expanded millions of fold and maintained in culture for more than 100 days without additional stimulation through their TCRs (FIG. 12). Furthermore, when the aforementioned target genes were simultaneously edited in healthy donor CD8⁺ T cells using CRISPR/Cas9, these edits reproducibly conferred a REX phenotype across different donors (FIG. 12).

### Example 9: Additional Genetic Edits for Clinical Profile

While the REX edits confer enhanced proliferation of the T_{REX} cell product, additional editing at the B2M and CD38 loci was performed to delay rejection of the T_{REX} cell product by a patient's immune system. B2M is a protein of 119 amino acids that is encoded by a gene on chromosome 15 in humans. It is also a component of major histocompatibility class (MHC) I molecules and also associates with non-classical, MHC I like molecules such as CD1, MR1, the neonatal Fc receptor, and Qa-1. Though it is located outside of the MHC locus, B2M is required for the successful expression of classical and non-classical MHC I molecules on the surfaces of nucleated cells. By eliminating B2M expression in T_{REX} cells using CRISPR/Cas9, the cells will be shielded from patient CD8+ T cells. Additionally, ablation of B2M expression and consequently MHC-I expression by the T_{REX} cell product will sensitize it to rejection by patient NK cells. Knockout of B2M was carried out simultaneously with knock in of a targeting a CAR (e.g. GPC3, HER2, BCMA) in the T_{REX} cell product.

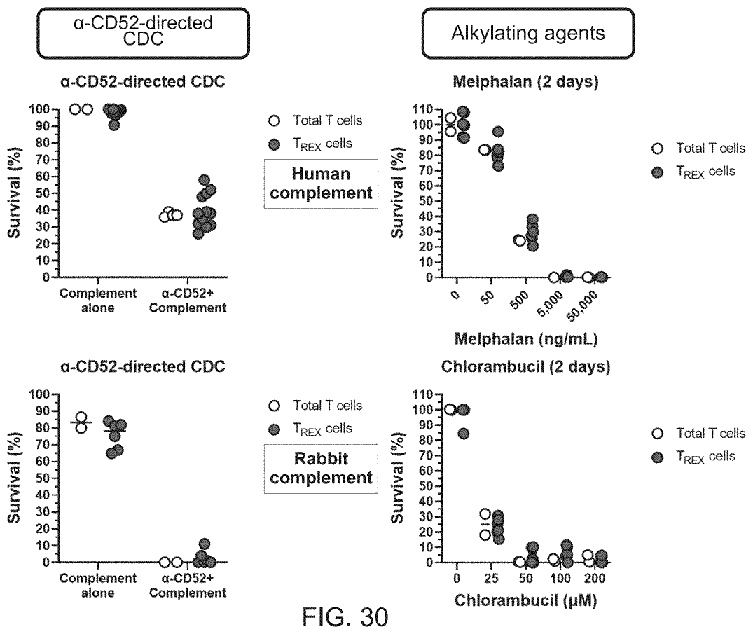

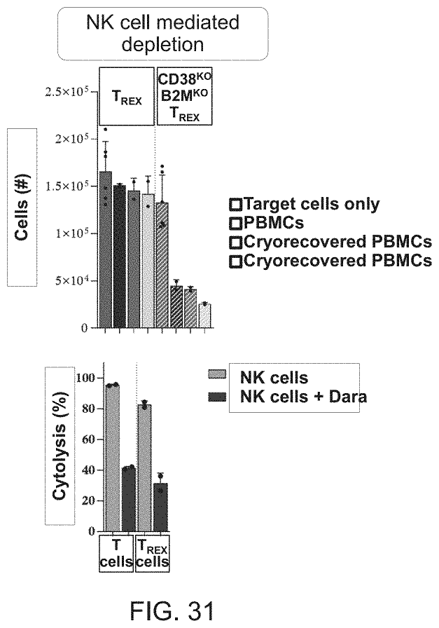

NK cells express high levels of CD38 and are depleted in certain cancer patients, e.g., multiple myeloma patients, receiving anti-CD38 monoclonal antibodies such as daratumumab and isatuximab. In order to prolong the persistence of this allogeneic cell population in patients, CD38 was knocked out of T_{REX} cells using CRISPR/Cas9 and daratumumab or isatuximab can be co-administered with T_{REX} cells (see FIGS. 30 and 31).

As an allogeneic CD8⁺ T cell population, T_{REX} cells are expected to be capable of targeting the HLA-mismatched patient's healthy cells through their TCRs, resulting in GvHD. In order to prevent the development of this pathology, the T_{REX} cell population was edited at the T Cell Receptor Alpha Constant (TRAC) locus, which encodes the TCR a chain. CRISPR/Cas9 editing of TRAC leads to loss of expression of the TCR a chain, which in turn prevents surface expression of the TCR by T_{REX} cells.

TABLE 4
Summary of cell edits

| Gene | Type of edit | Purpose |
|---|---|---|
| CDKN2A | CRISPR/Cas9 deletion | Resistance to replicative senescence |
| CDKN2B | CRISPR/Cas9 deletion | Resistance to replicative senescence |
| MTAP [symbol] | CRISPR/Cas9 deletion | Resistance to replicative senescence |
| B2M | CRISPR/Cas9 deletion | Limit HVG |
| TRAC | CRISPR/Cas9 deletion | Avoid GvHD |
| CD38 | CRISPR/Cas9 deletion | Resistance to daratumumab |

### Example 10: Anti BCMA-T_{REX} Allogeneic Cell Therapy

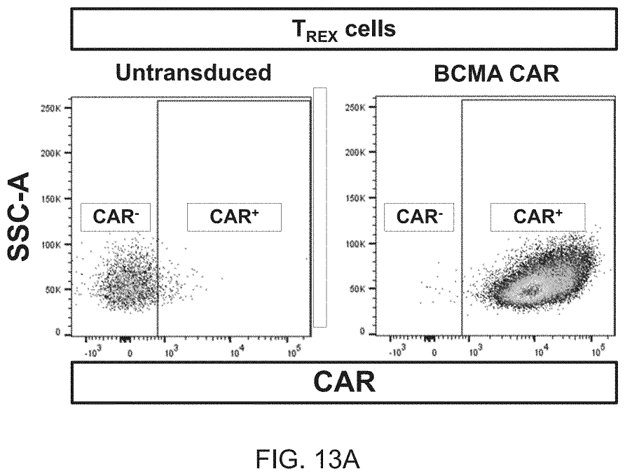

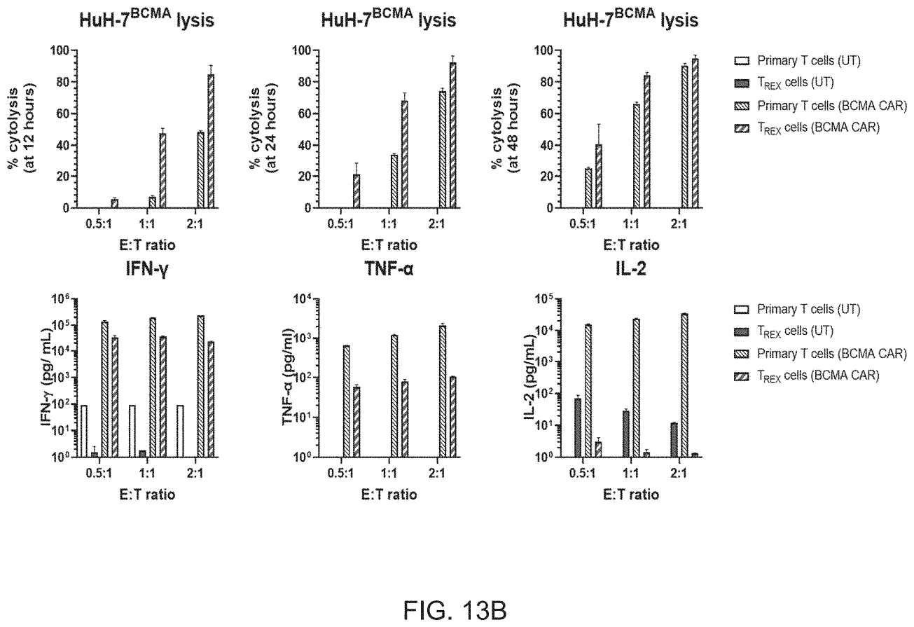

A BCMA targeting CAR was expressed in T_{REX} cells (i.e., cells lacking CDKN2A/CDKN2B/MTAP) (FIG. 13A). The genome of the anti-BCMA-T_{REX} cells was further edited to ablate expression of Human Leukocyte Antigen (HLA) class I and the αβ T cell receptor (TCR) by inactivation of the B2M and TRAC genes, respectively, to minimize host-versus-graft (HvG) and graft-versus-host (GvH) allogeneic responses, respectively. Additionally, the CD38 gene was inactivated in anti-BCMA-T_{REX} cells using CRISPR/Cas9 to render the cells resistant to anti-CD38 depleting monoclonal antibodies. The inactivation of these three genes enhances the total in vitro expansion potential of peripheral blood CD8⁺ T cells such that downstream cell population numbers far exceed those achievable with unedited peripheral blood CD8⁺ T cells. The cells retain hallmark proliferative characteristics of primary T cells (dependence on both anti-CD3 stimulation prior to TRAC inactivation and IL-2 for expansion/survival) but with greater potential for expansion. Anti-BCMA-T_{REX} cells maintain cytotoxic function but display reduced cytokine release compared to conventional CAR-T cell preparations composed of mixed CD4⁺ and CD8⁺ T cell populations.

Assessment of anti-BCMA-T_{REX} cells indicates that these cells are likely to control BCMA-expressing tumors similarly to primary anti-BCMA-CAR-T cells while exhibiting a potentially improved safety profile in the form of diminished cytokine release and potentially reduced risk of CRS (FIG. 13B). Briefly, anti-BCMA-T_{REX} cells and anti-BCMA-CAR-T cells were cultured with BCMA-expressing tumor cells. Tumor cell lysis was measured at varying effector:target cell ratios at different timepoints following initiation of co-culture (FIG. 13B, top row). Supernatants were collected 72 hours after start of co-culture and levels of IFN-γ, TNF-α, and IL-2 were determined (FIG. 13B, bottom row) by MSD.

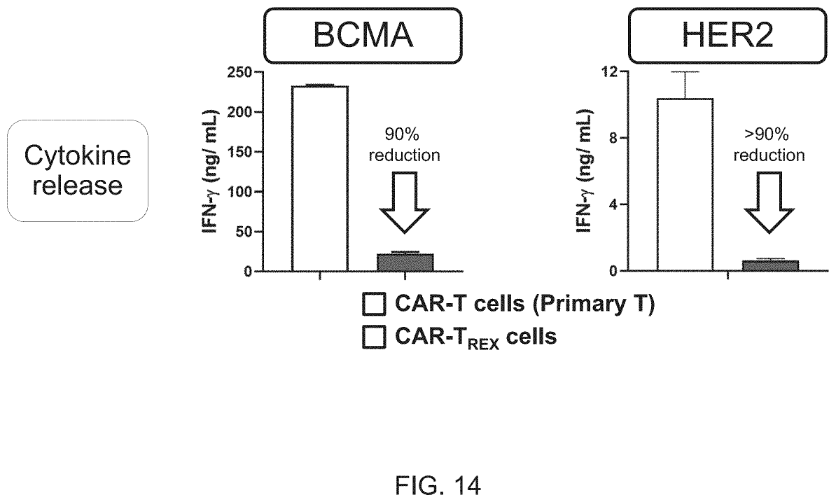

Anti-BCMA-T_{REX} cells (82 days in culture) or anti-BCMA-CAR-T cells were cultured with BCMA-expressing tumor cells. Supernatants were collected 72 hours after initiation of co-culture and assessed for levels of IFN-γ using MSD kits (FIG. 14, left). Data demonstrate a 90% reduction in IFN-γ levels in co-cultures with anti-BCMA-T_{REX} cells (83 days in culture) than in co-cultures with anti-BCMA-CAR-T cells despite similar control of tumor cells. These data demonstrate that CAR-T_{REX} cells exhibit a cytokine secretion profile that may confer lower risk of CRS than CAR-T cells.

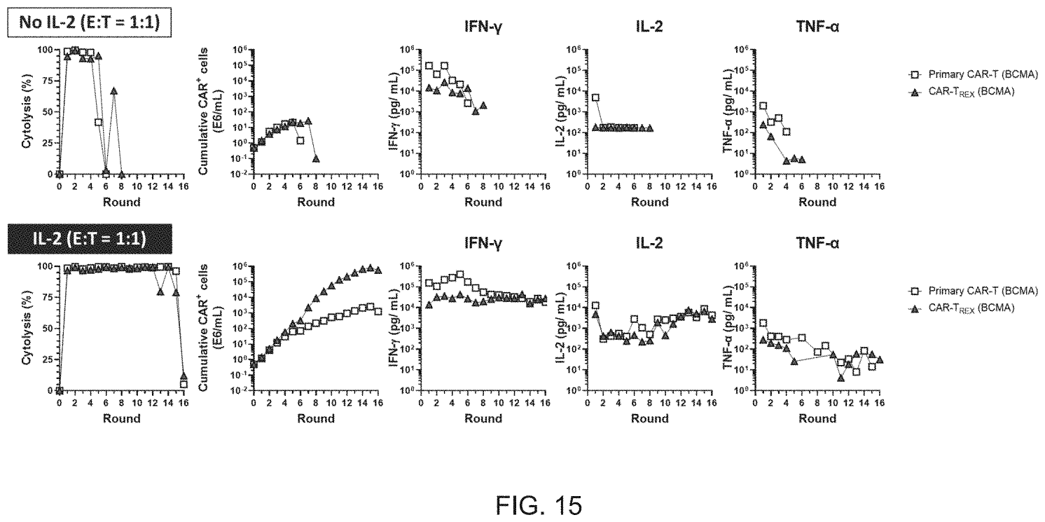

Anti-BCMA-T_{REX} cells (112 days in culture) were assessed for their ability to persist in a serial kill assay with or without IL-2 support. Briefly, anti-BCMA-T_{REX} cells or anti-BCMA-CAR-T cells were serially cultured with BCMA-expressing JJN3 cells at an effector:target cell ratio of 1:1. Tumor cell control (% cytolysis), effector cell numbers, and effector cytokine secretion was measured after each round of co-culture and graphed (FIG. 15). Anti-BCMA-T_{REX} cells persisted a comparable number of rounds in this serial kill assay as anti-BCMA-CAR-T cells and inclusion of IL-2 in the cell culture medium further increased the number of rounds for which anti-BCMA-T_{REX} cells and anti-BCMA-CAR-T cells could control tumor cell growth. Anti-BCMA-T_{REX} cells and anti-BCMA-CAR-T cells demonstrated enhanced proliferation in response to IL-2 and effector cytokine secretion was sustained for a longer duration in co-cultures in which IL-2 was included in the culture medium (FIG. 15 top versus bottom rows). These data indicate that anti-BCMA-T_{REX} cells demonstrate similar cytotoxicity to anti-BCMA-CAR-T cells in vitro and also exhibit a similar capacity to respond to exogenous IL-2. Further, anti-BCMA-T_{REX} cells secreted lower levels of effector cytokines following CAR-engagement than anti-BCMA-CAR-T cells despite comparable tumor control.

### Example 11: Anti HER2-T_{REX} Allogeneic Cell Therapy

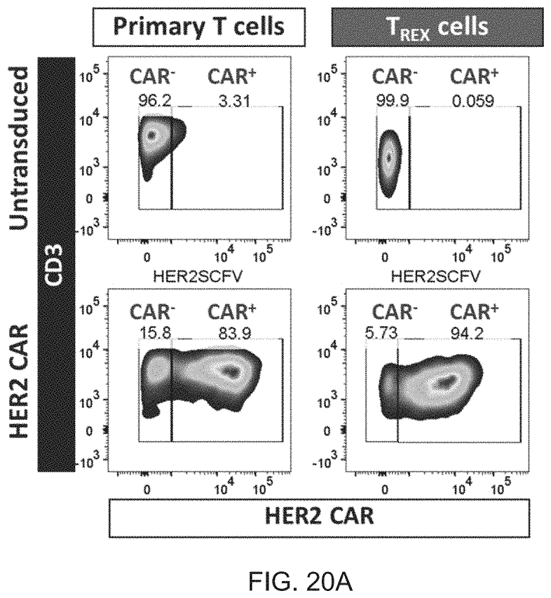

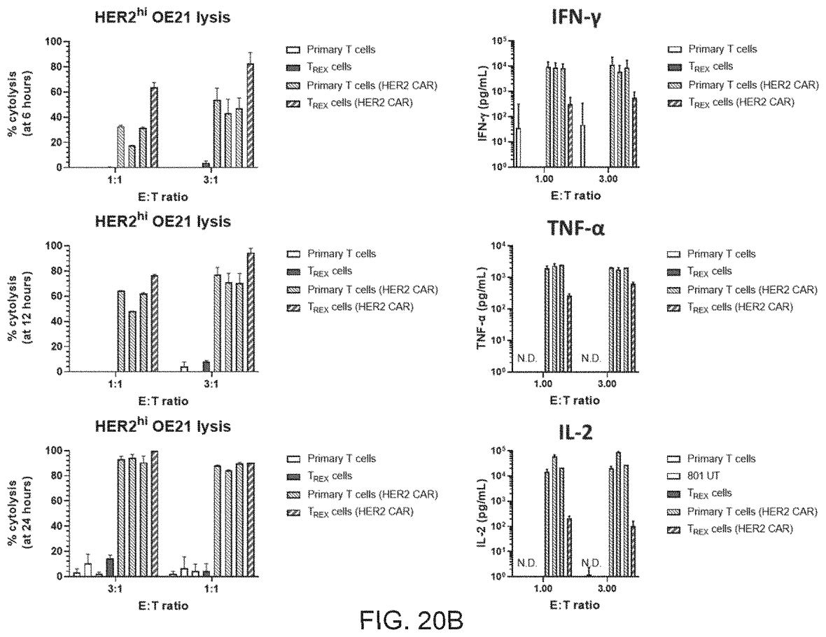

A HER2 targeting CAR was expressed in T_{REX} cells or Primary T cells (FIG. 20A) to generate CAR-T_{REX} cells and CAR-T cells. Anti-HER2-T_{REX} cells and anti-HER2-CAR-T cells were assessed for their ability to target HER2 overexpressing OE21 cells at varying effector:target cell ratios (FIG. 20B, left). Anti-HER-T_{REX} cells demonstrated comparable or improved control of HER2-expressing tumor cells relative to anti-HER2-CAR-T cells generated from three different Primary T cell donors. Supernatants were collected 72 hours after initiation of co-culture and subsequently examined for the presence of effector cytokines (FIG. 20B, right). As was previously observed, despite comparable or improved tumor cell control, anti-HER2-T_{REX} cells secreted lower levels of cytokines (IFN-γ, TNF-α, and IL-2) than anti-HER2-CAR-T cells, suggesting that CAR-T_{REX} cells may have a lower propensity to cause CRS in patients. Additionally, as shown above for anti-BCMA-T_{REX} cells, reduced secretion of IFN-γ was also observed in supernatants taken from co-cultures of HER2-expressing tumor cells and anti-HER2-T_{REX} cells as compared with supernatants from co-cultures with anti-HER2-CAR-T cells (FIG. 14, right). These data again demonstrate that CAR-T_{REX} cells exhibit a cytokine secretion profile that may confer lower risk of CRS than CAR-T cells.

### Example 12: T_{REX} Cell Phenotype can be Generated Using Different Combinations of Edits

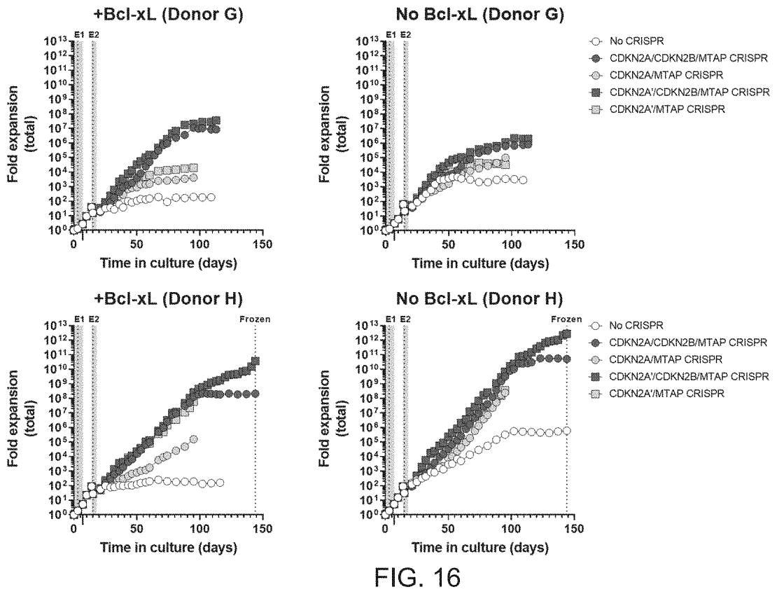

Requirements for overexpression of Bcl-xL and the various REX target genes to confer the REX phenotype were assessed in isolated CD8⁺ T cells from two donors (denoted as G and H). Briefly, CD8⁺ T cells were negatively selected and then activated with αCD3/αCD28 Dynabeads for 3 days. Bcl-xL was introduced into some cells while other cells were cultured and various combinations of the REX target genes were knocked out using CRISPR/Cas9 (FIG. 16). Cell expansion was monitored and graphed over time. (CDKN2A and CDKN2A′ reflect targeting of single versus multiple isoforms). Bcl-xL was found to be dispensable for the REX phenotype while the three REX target genes yielded a consistent phenotype across donors (FIG. 16, right).

### Example 13: T_{REX} Cells are Edited at the Targeted Loci

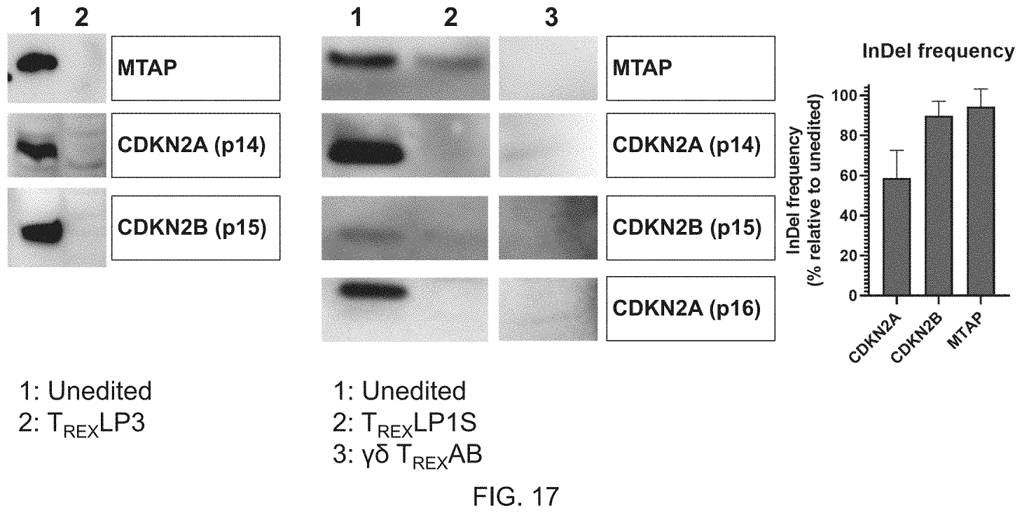

T_{REX} cells and γδ T_{REX} cells were examined for ablation of expression of the REX target genes by Western Blot analysis (FIG. 17, left and center panels). As expected these cells demonstrated loss of expression of MTAP, CDKN2A (p14), CDKN2A (p16), and CDKN2B (p15). In contrast, expression of these genes was maintained in donor-matched, unedited control cells. Further, Sanger Sequencing data indicate a high prevalence of InDels at these three loci in edited T_{REX} cells (FIG. 17, right).

### Example 14: T_{REX} Cells Demonstrate Enrichment in Cell Cycle-Associated Gene Signatures

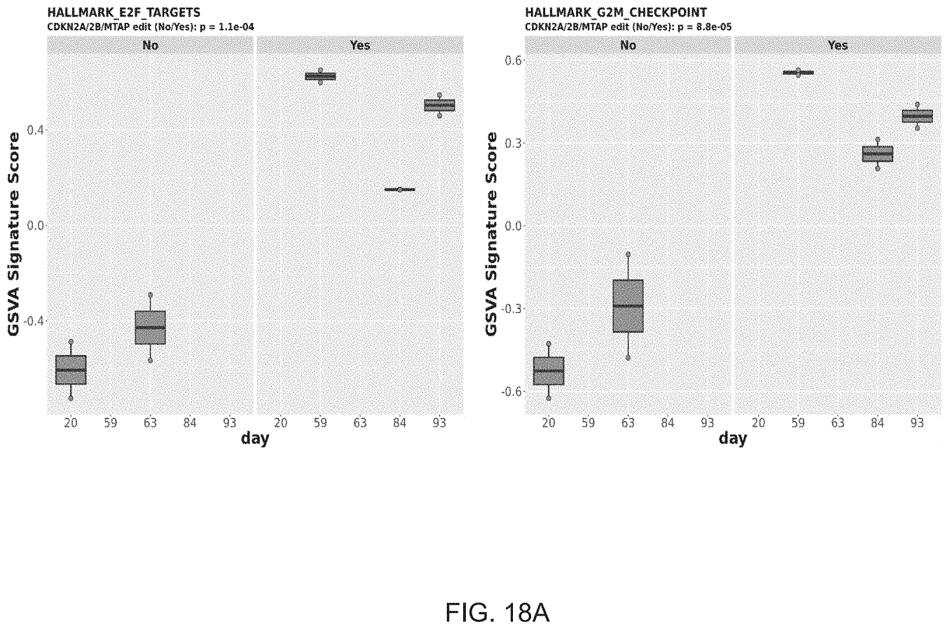

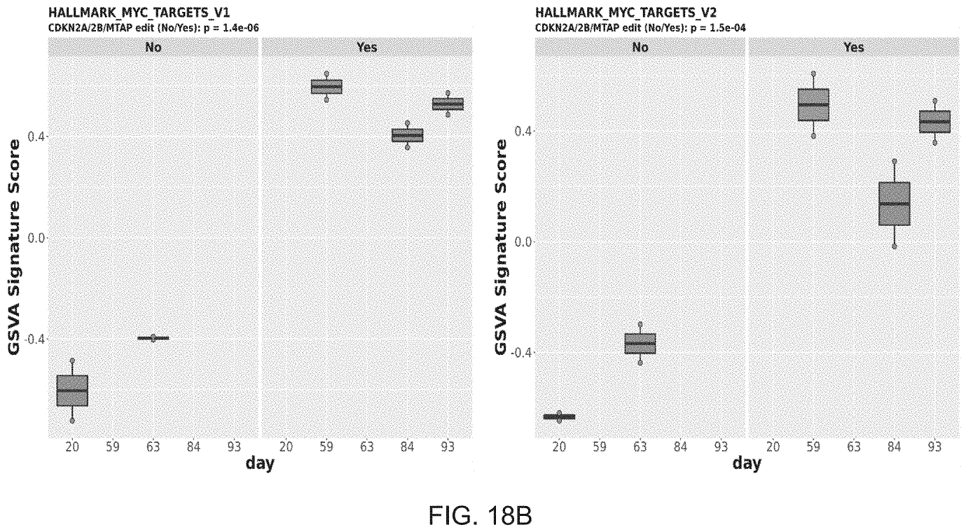

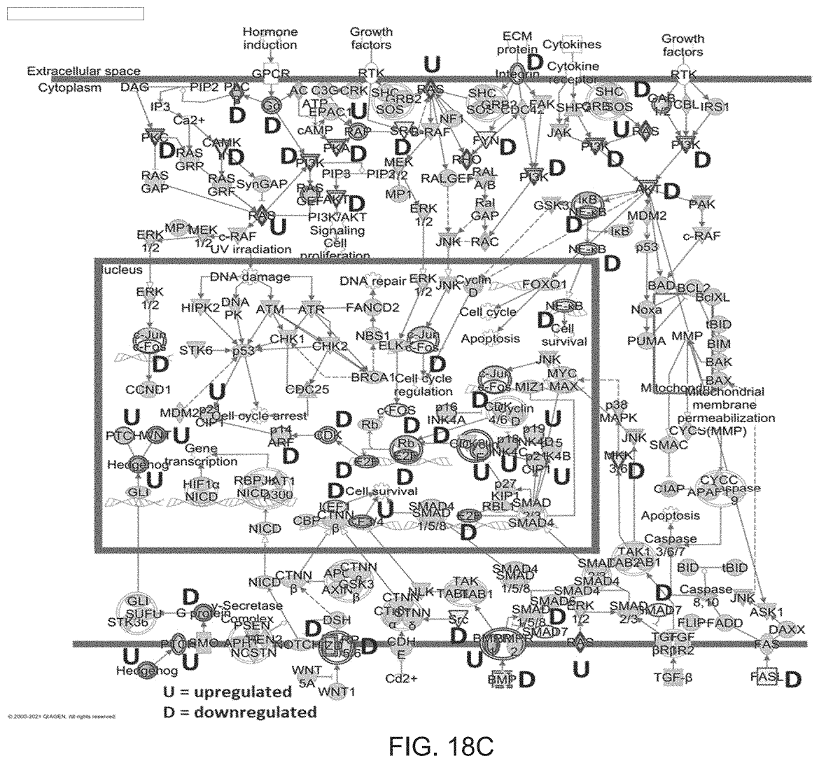

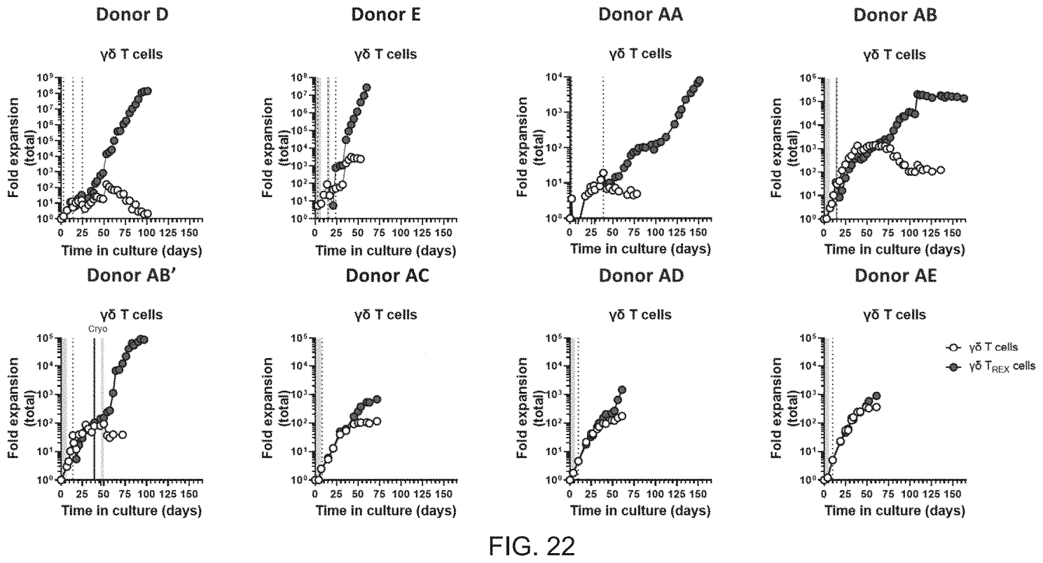

Bcl-xL overexpressing T_{REX} cells and donor-matched unedited control CD8⁺ T cells were cultured over time. RNAseq analysis was performed on cell pellets generated at various points and gene signatures were assessed in Bcl-xL T_{REX} cells and control cells; as expected, TREX cells showed enrichment in gene signatures associated with cell cycle such as E2F target genes and G2M checkpoint target genes (FIG. 18A). Bcl-xL T_{REX} cells also showed higher levels of expression of MYC target genes (FIG. 18B) in agreement with the observed proliferation rates of these cells. Further, Bcl-xL T_{REX} cells showed modulation of expression of multiple cell-cycle-associated genes (FIG. 22C). These data confirm that the REX phenotype is associated with cell cycle progression and increased proliferation.

### Example 15: T_{REX} Cells are Dependent on IL-2 for Survival and Proliferation

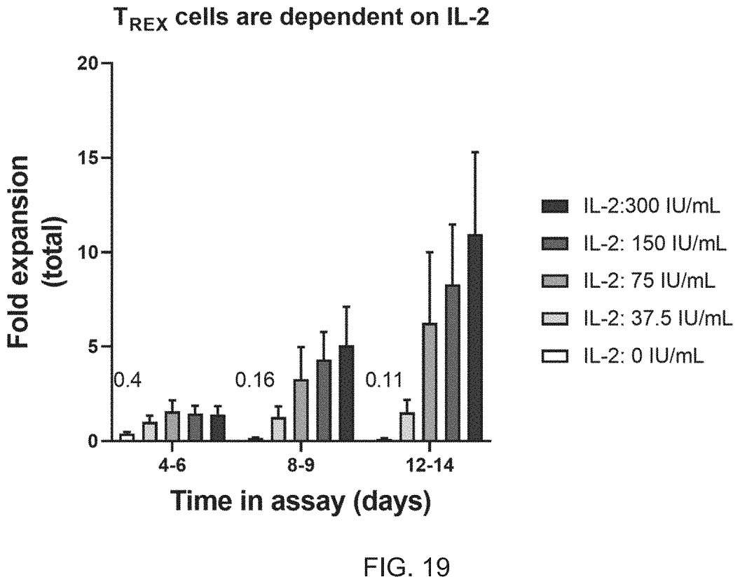

T_{REX} cells and were cultured with varying amounts of IL-2 for a period of 12-14 days. Cell expansion was tracked and graphed over this time (FIG. 19). As shown above for Bcl-xL T_{REX} cells (see, e.g., FIG. 5), T_{REX} cells are highly dependent on IL-2 for proliferation and survival in vitro. T_{REX} cells exhibited dose-dependent proliferation in response to IL-2; in the absence of IL-2, T_{REX} cells showed a rapid decline in survival with more than 60% of T_{REX} cells being eliminated within the first 4 days.

### Example 17: REX Edits Bolster the Proliferative Capacity of CD4+T_{REX} Cells

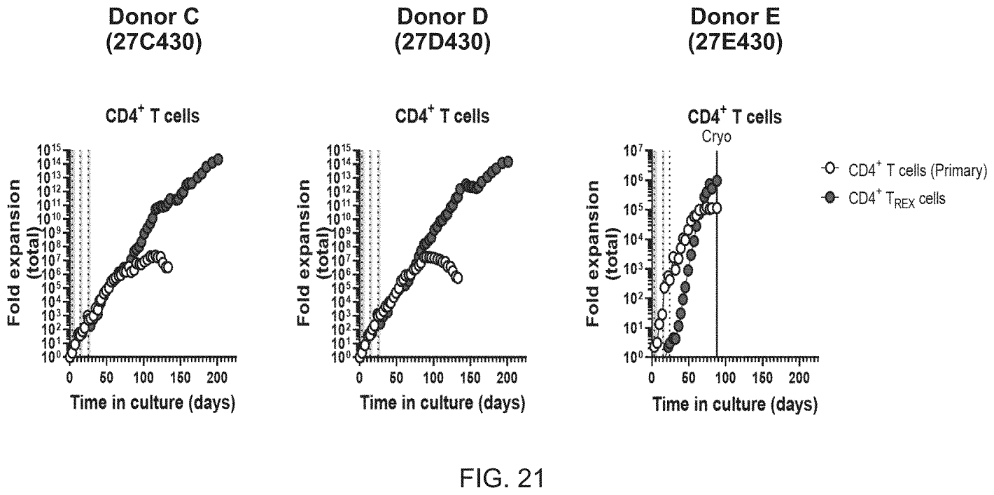

REX edits reproducibly confer an enhanced resistance to replicative senescence in CD8⁺ T cells. The capacity of ablation of expression of the REX target genes (CDKN2A, CDKN2B, and MTAP) to enhance CD4⁺ T cell resistance to replicative senescence was determined. CD4⁺ T cells were isolated from three healthy donors, stimulated using αCD3/αCD28 Dynabeads and then edited at these loci. Proliferation of CD4⁺ T_{REX} cells and donor-matched unedited CD4⁺ T cell controls was tracked over time and graphed. (FIG. 21). As was previously demonstrated with CD8⁺ T cells, targeting the REX genes in CD4⁺ T cells reproducibly bolstered the proliferative capacity of these cells and rendered them resistant to replicative senescence.

### Example 18: γδ T_{REX} Cells can be Generated Using REX Edits

γδ T cells are another cytotoxic subset of T cells. The ability of REX edits to confer a T_{REX} cell phenotype in γδ T cells was investigated using γδ T cells from eight different donors (FIG. 22). γδ T cells were isolated and stimulated with αCD3/αCD28 Dynabeads or αCD3 antibody and subsequently edited at the REX loci using CRISPR/Cas9. γδ T cell and γδ T_{REX} cell proliferation was monitored and graphed over time. REX edits reproducibly enhanced γδ T cell resistance to replicative senescence and led to generation of a γδ T_{REX} cell phenotype.

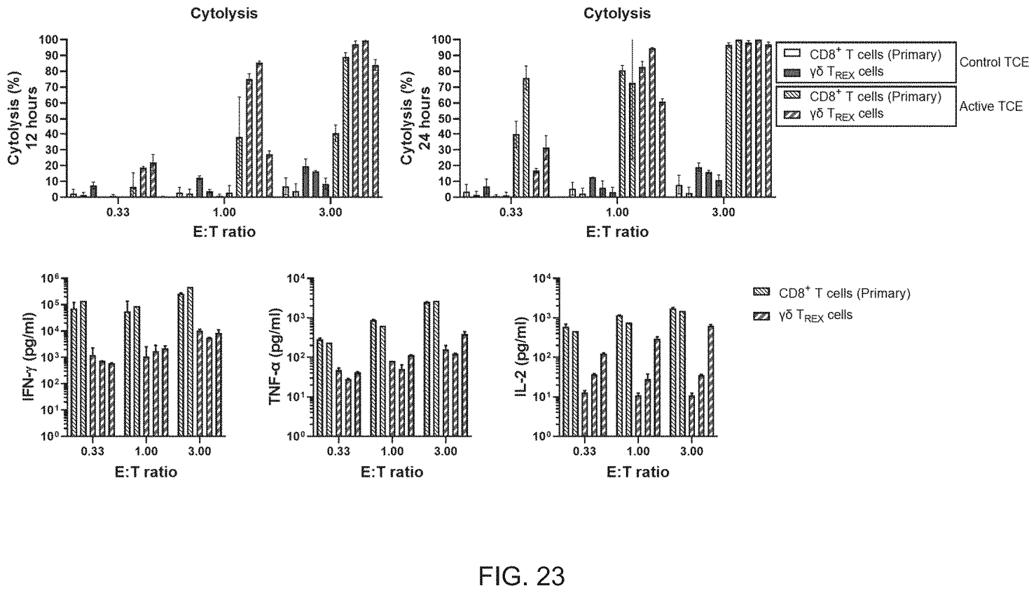

Having established that γδ T_{REX} cell lines demonstrate enhanced resistance to replicative senescence, it was determined whether γδ T_{REX} cells maintain potent cytotoxic function after long-term culture and expansion. A T cell engager was used in the presence of target tumor cells and the impedance-based xCELLigence platform to quantify γδ T_{REX} cell cytotoxic function (FIG. 23). Day 79 and Day 88 γδ T_{REX} cell lines demonstrated a comparable ability to lyse target tumor cells in the presence of the T cell engager as unmodified primary CD8⁺ T cells (FIG. 23, top). Supernatants from these co-cultures were collected 72 hours after the addition of effector cells and the active T cell engager or control T cell engager molecule and analyzed for the presence of IFN-γ, IL-2, and TNF-α (FIG. 23, bottom). γδ T_{REX} cell lines produced lower levels of these cytokines than unmodified primary CD8⁺ T cells despite a similar ability to lyse target cells in an antigen-dependent manner. These data indicate that even after 79 days in culture and substantial expansion, γδ T_{REX} cells are not functionally exhausted and maintain their cytotoxic potential.

γδ T cells are typically comprised of multiple subsets including Vδ1, Vδ2, Vδ3, and Vδ5, among others (Lawand et al., Front. Immunol., 30 Jun. 2017). In humans the Vδ1 and Vδ2 make up the majority of γδ T cells with Vδ2 cells being found primarily in the blood and VM cells being found in tissues.

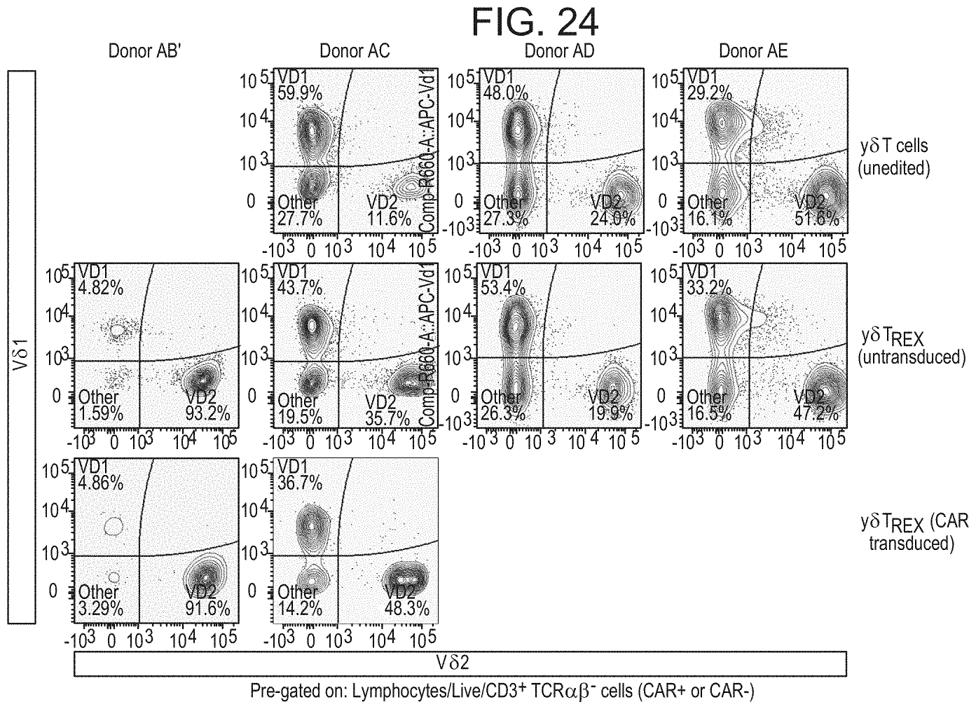

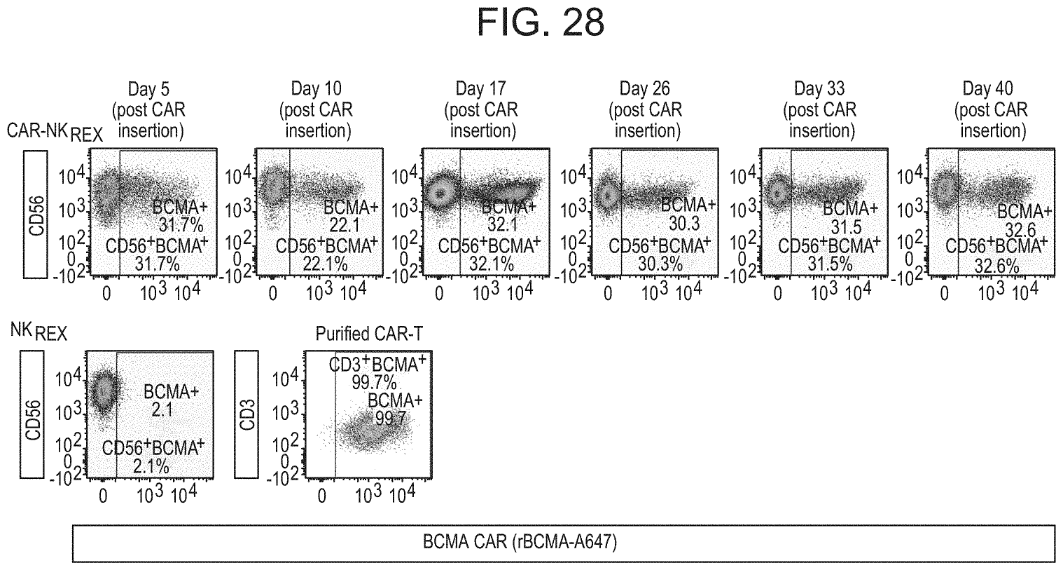

γδ T_{REX} cells, γδ CAR-T_{REX} cells, and donor-matched, unedited γδ T cells were stained and analyzed for expression of Vδ1 and Vδ2 (FIG. 24). FACS analysis revealed that γδ T_{REX} cells were comprised of multiple γδ T cell subtypes (Vδ1, Vδ2, and Vδ1⁻Vδ2⁻), indicating that REX edits can enhance resistance to replicative senescence for multiple γδ T cell subtypes. Further, diversity of γδ T cell subtypes was maintained in γδ CAR-T_{REX} cells (FIG. 28, bottom).

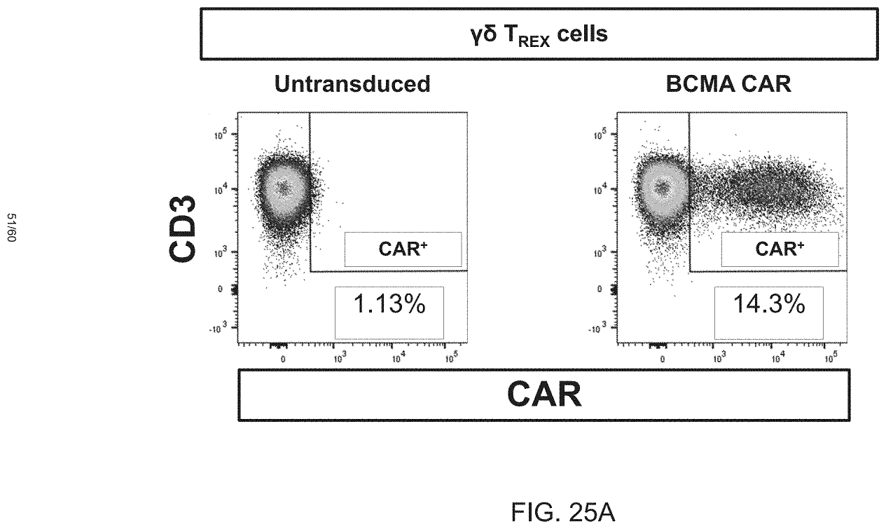

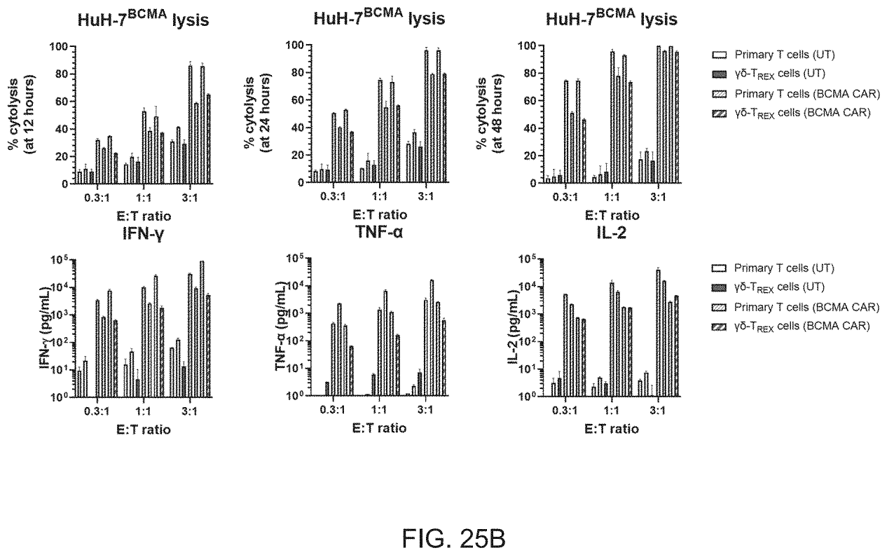

γδ T_{REX} cells were next investigated for their ability take instruction from a tumor-targeting moiety such as a BCMA-targeting CAR (FIG. 25). γδ T_{REX} cells were transduced to express a BCMA-targeting CAR (FIG. 25A), and these cells were co-cultured with BCMA-expressing tumor cells at various effector:target cell ratios. Tumor cell lysis was monitored over time using the xCELLigence platform (FIG. 25B) and supernatants were harvested 72 hours after initiation of co-culture. γδ T_{REX} cells demonstrated similar ability to control BCMA-expressing tumor cells as primary CAR-T cell controls, however, they generally secreted lower levels of effector cytokines including IFN-γ, TNF-α, and IL-2 (FIG. 25B, bottom). These data indicate γδ CAR-T_{REX} cells are capable of taking direction from a tumor-targeting CAR and may also be less likely than Primary CAR-T cells to cause CRS.

### Example 19: REX Edits in NK Cells Support an NK_{REX} Cell Phenotype

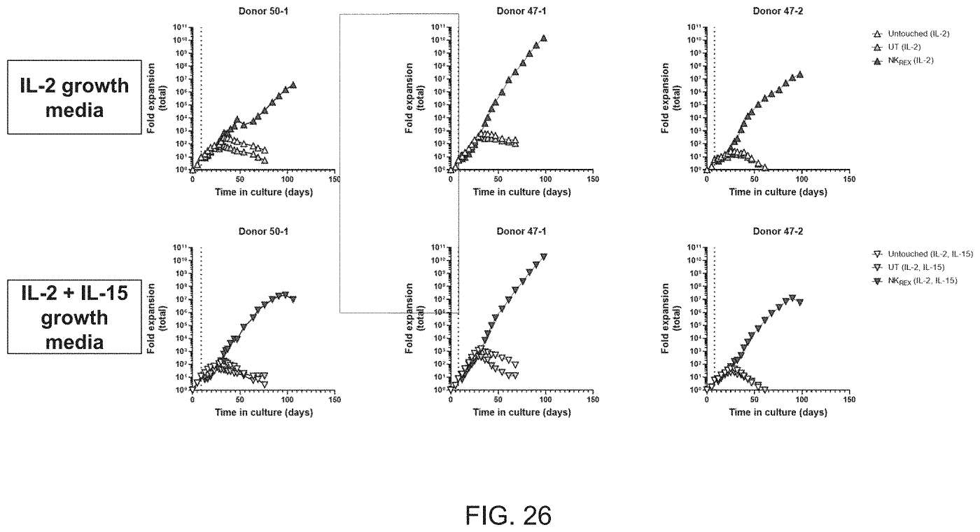

REX edits enhance T cell resistance to replicative senescence, however it was unclear whether they would support an NK_{REX} cell phenotype. Therefore, NK cells were isolated from three different donors and cultured in media containing IL-2 or a combination of IL-2 and IL-15. NK cells were then edited at the REX loci using CRISPR/Cas9 and proliferation of NK_{REX} and donor-matched unedited NK cells was monitored over time (FIG. 26). In all donors and cytokine conditions, REX edits were reproducibly able to enhance NK_{REX} cell resistance to replicative senescence (FIG. 26). NK_{REX} cells could be cultured for over 90 days, expanding >10⁶->10¹⁰ fold while unedited NK cells failed to expand and died within 80 days.

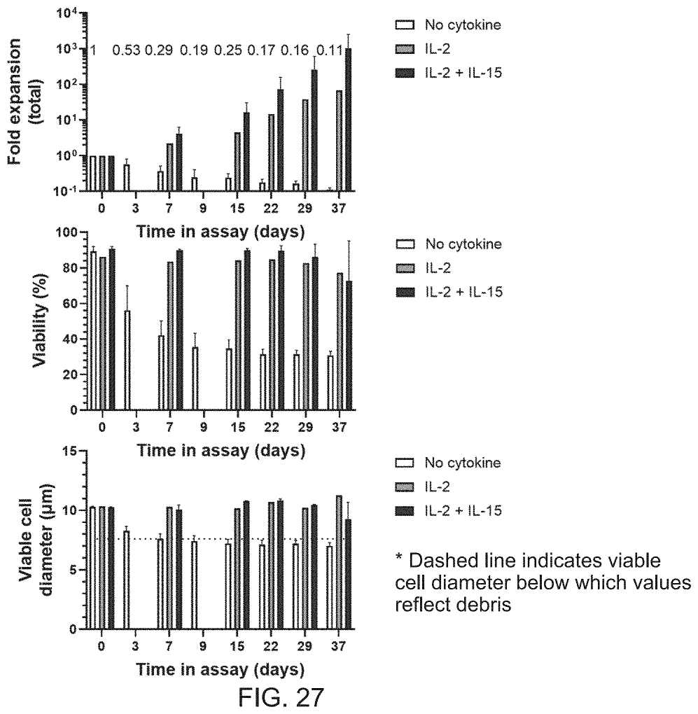

Given the enhancement in resistance to replicative senescence, it was important to determine whether NK_{REX} cells maintained their dependence on cytokine support. NK_{REX} cells were generated in media containing IL-2 or a combination of IL-2 and IL-15. NK_{REX} cell dependency on these cytokines was determined in an experiment in which cytokines were withdrawn from the growth media and NK_{REX} cell numbers were monitored for a period of 37 days. NK_{REX} cells failed to proliferate following cytokine withdrawal and these cells demonstrated a rapid drop off in cell viability and viable cell diameter, reinforcing their dependency on cytokine support despite editing of the REX genes (FIG. 27).

NK_{REX} cells were transduced to express a BCMA-targeting CAR to determine whether these cells were capable of stably expressing a tumor-targeting CAR (FIG. 28). CAR expression was maintained in CAR-NK_{REX} cells over time and levels of expression (mean fluorescence intensity, MFI) was similar to that in purified CAR-T cells. These data indicate that CAR-NK_{REX} cells can stably express a CAR and that levels of expression are comparable to that of standard CAR-T cells.

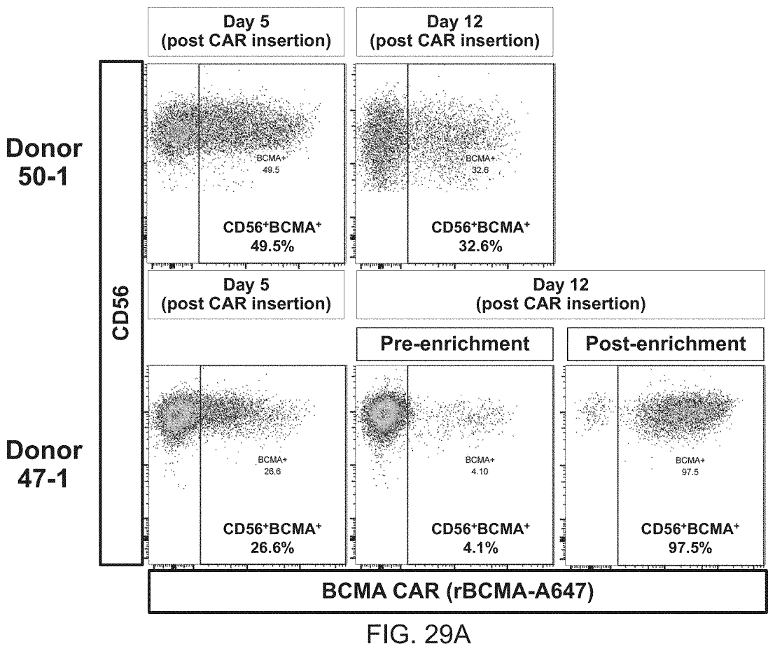

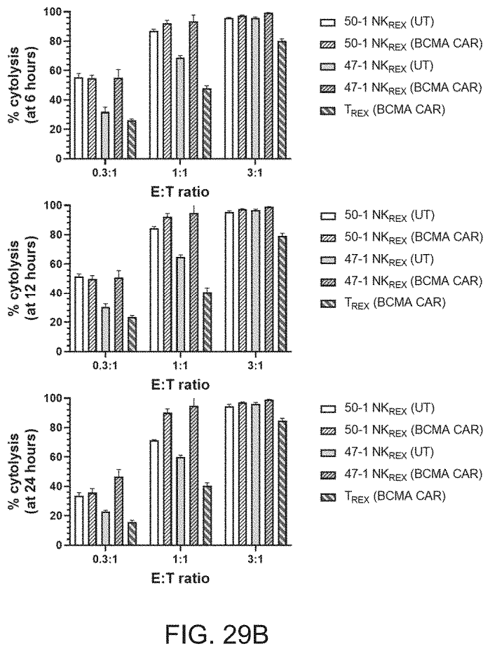

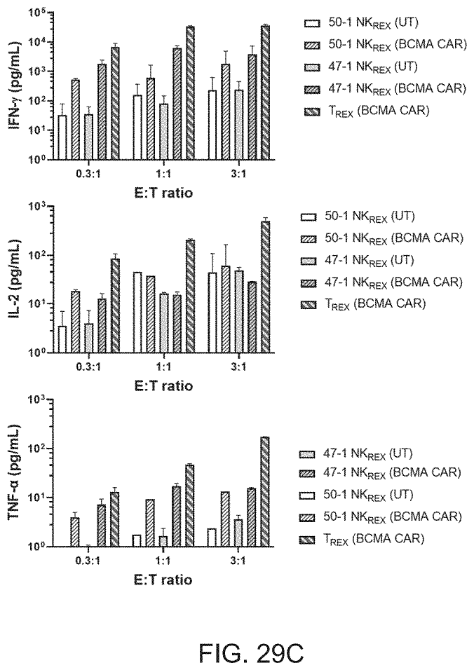

While NK_{REX} cells could be expanded in culture for long periods of time, it was unclear: 1) whether their cytotoxic potential was maintained following sustained proliferation; and 2) whether they could take direction a tumor-targeting CAR. Therefore, CAR-NK_{REX} cells were generated from two different NK_{REX} lines (FIG. 29A). One of these CAR-NK_{REX} lines was purified based on CAR expression to generate a >95% CAR′ CAR-NK_{REX} line (FIG. 29A, bottom right). NK_{REX} and CAR-NK_{REX} lines from donors 50-1 and 47-1 were tested for their ability to lyse BCMA-expressing tumor cells in an xCELLigence assay (FIG. 29B). Even after 78 and 86 days in culture, NK_{REX} and CAR-NK_{REX} cells were potently cytotoxic. These lines rapidly lysed BCMA-expressing target cells, achieving a higher level of control more rapidly than CAR-T_{REX} cells (FIG. 29B). While NK_{REX} cells were able to lyse tumor cells independently of CAR expression, likely due to engagement of activating receptors on NK_{REX} and CAR-NK_{REX} cells, at lower effector:target cell ratios, contributions of CAR-directed cytotoxicity could be observed for both donors 50-1 and 47-1. Supernatants were collected after 48 hours of co-culture and levels of IFN-γ, IL-2, and TNF-α were determined using MSD (FIG. 29C). NK_{REX} cells secreted lower levels of these cytokines than CAR-NK_{REX} cells, which CAR-T_{REX} cells secreted the highest levels of these factors (FIG. 29C). These data indicate that CAR-NK_{REX} cells are capable of stably expressing and taking direction from a tumor-targeting CAR. Further, these cells rapidly lyse tumor cells and accumulate lower levels of IFN-γ, IL-2, and TNF-α in co-culture supernatants.

### Example 20: T_{REX} Cells are Sensitive to T Cell Depleting Agents and Chemotherapies

T^{REX} cells have been modified to increase their resistance to replicative senescence. However, these cells have displayed hallmarks of normal T cells. In order to better understand the ability to control T_{REX} cells, their susceptibility to standard T cell depleting agents and chemotherapies was determined relative to unedited total T cells that were activated to enter cell cycle (FIG. 30). Unedited, recently activated total T cells or T_{REX} cells were incubated with 10 μg/mL anti-CD52 and 10% human complement (FIG. 30, top left) or 10% rabbit complement (FIG. 30, bottom left). After 3 hours, cell survival was assessed using a Cell Titer Glo assay. Unedited, recently activated total T cells or T_{REX} cells were also incubated with indicated quantities of melphalan (FIG. 30, top right) or chlorambucil (FIG. 30, bottom right) and cell survival was measured after 2 days using a Cell Titer Glo assay. In all cases, T_{REX} cells demonstrated comparable susceptibility to these agents as unedited, recently activated total T cells.

### Example 21: B2M^{KO} T_{REX} Cells are Sensitive to NK Cell Mediated Depletion and this can be Modulated Using Anti-CD38 Antibodies

As an allogenic cell product, T_{REX} cells are modified at the B2M locus, increasing their susceptibility to NK cell mediated depletion. These cells can be further modified at the CD38 locus to limit their depletion by anti-CD38 antibodies. T_{REX} cell lines and CD38^{KO}B2M^{KO} T_{REX} cell lines were generated using CRISPR/Cas9. T_{REX} cells and CD38^{KO}B2M^{KO} T_{REX} cells were co-cultured with PBMCs isolated from healthy donors. T_{REX} cells did not exhibit a drop in number when co-cultured with PBMCs while CD38^{KO}B2M^{KO} T_{REX} cells were susceptible to NK cell mediated lysis as expected (FIG. 31, top). NK cells express high levels of CD38 and when NK cells were preincubated with the CD38 targeting antibody Daratumamab (Dara) prior to co-culture with CD38^{KO}B2M^{KO} total T cells or CD38^{KO}B2M^{KO} T_{REX} cells, this conferred a >50% reduction in cytolysis. These data indicate that CD38^{KO}B2M^{KO} T_{REX} cells are susceptible to NK cell mediated lysis and that this sensitivity to depletion can be regulated through the administration of anti-CD38 antibodies.

1. A method of generating a population of primary immune cells resistant to replicative senescence (RRS), comprising:
(a) introducing one or more genetic edits to primary immune cells; and
(b) culturing the primary immune cells in a culture medium;
wherein the culturing induces proliferation of the primary immune cells to yield a population of primary immune cells resistant to replicative senescence (RRS).

2.-4. (canceled)

5. The method of claim 4, wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP).

6.-7. (canceled)

8. The method of claim 1, wherein introducing one or more genetic edits comprise introducing one or more transgenes encoding an anti-apoptotic factor or a virally-derived factor into the primary immune cells.

9. The method of claim 8, wherein the anti-apoptotic factor is either B-cell lymphoma-extra large (Bcl-xL) or B-cell lymphoma 2 (Bcl-2).

10.-11. (canceled)

12. The method of claim 1 further comprising inhibiting the expression of phosphatase and tensin homolog (PTEN).

13.-26. (canceled)

27. A method of generating a population of primary immune cells resistant to replicative senescence (RRS), comprising:
(a) inhibiting the expression of one or more endogenous regulatory factors in the primary immune cells,
wherein the endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP);
(b) inhibiting the expression of one or more endogenous immune related genes in the primary immune cells,
wherein the endogenous immune related gene is beta-2 microglobulin (B2M), and/or T-cell receptor α constant (TRAC); and
(c) culturing the primary immune cells in a culture medium;
wherein the culturing induces proliferation of the primary immune cells to yield a population of primary immune cells resistant to replicative senescence (RRS).

28. The method of claim 27 further comprising introducing a transgene encoding either B-cell lymphoma-extra large (Bcl-xL) or B-cell lymphoma 2 (Bcl-2) into the primary immune cells.

29.-31. (canceled)

32. The method of claim 27 further comprising inhibiting the expression of phosphatase and tensin homolog (PTEN).

33.-46. (canceled)

47. A method of generating a population of primary immune cells resistant to replicative senescence (RRS), comprising:
(a) inhibiting expression of one or more endogenous regulatory factors in the primary immune cells,
wherein endogenous regulatory factor is cyclin-dependent kinase inhibitor 2A (CDKN2A), cyclin-dependent kinase inhibitor 2B (CDKN2B), or S-methyl-5′-thioadenosine phosphorylase (MTAP); and
(b) culturing the primary immune cells in a culture medium;
wherein the culturing induces proliferation of the primary immune cells to yield a population of primary immune cells resistant to replicative senescence (RRS).

48.-52. (canceled)

53. The method of claim 47 further comprising inhibiting the expression of phosphatase and tensin homolog (PTEN).

54.-67. (canceled)

68. An engineered immune cell population produced according to the method of claim 1.

69. A pharmaceutical composition comprising the engineered immune cell population of claim 68 and a pharmaceutically acceptable carrier.

70. method of treating a cancer in a subject in need thereof, comprising administering to the subject a therapeutically effective amount of the pharmaceutical composition of claim 69.

71. An engineered T cell that does not express cyclin-dependent kinase inhibitor 2A (CDKN2A), cyclin-dependent kinase inhibitor 2B (CDKN2B), and/or S-methyl-5′-thioadenosine phosphorylase (MTAP).

72. The engineered T cell of claim 71, wherein the engineered T cell further comprises a transgene encoding either B-cell lymphoma-extra large (Bcl-xL) or B-cell lymphoma 2 (Bcl-2).

73. The engineered T cell of claim 71, wherein the engineered T cell does not express of one or more endogenous immune related genes in the primary immune cells.

74. The engineered T cell of claim 73, wherein the endogenous immune related gene is beta-2 microglobulin (B2M), and/or T-cell receptor α constant (TRAC).

75. The engineered T cell of claim 71 wherein the engineered T cell does not express cluster of differentiation 38 (CD38).

76. The engineered T cell of claim 71 further comprising a polynucleotide that encodes a chimeric antigen receptor (CAR).

77. The engineered T cell of claim 71, wherein the engineered T cell is a CD8⁺ T cell, a CD4⁺ T cell, a gamma-delta T cell, a mucosal associated invariant T (MAIT) T cell, a natural killer (NK) cell, a natural killer T (NKT) cell, or a combination thereof.

78. The engineered T cell of claim 71, wherein the engineered T cell is a CD8⁺ T cell.

79. The engineered T cell of claim 71, wherein the engineered T cell is a CD4⁺ T cell.

80. The engineered T cell of claim 71, wherein the engineered T cell is human.

81. An engineered T cell that does not express cyclin-dependent kinase inhibitor 2A (CDKN2A), cyclin-dependent kinase inhibitor 2B (CDKN2B), S-methyl-5′-thioadenosine phosphorylase (MTAP), beta-2 microglobulin (B2M), and/or T-cell receptor α constant (TRAC).

82. The engineered T cell of claim 81, wherein the engineered T cell does not express cluster of differentiation 38 (CD38).

83. The engineered T cell of claim 81 further comprising a polynucleotide that encodes a chimeric antigen receptor (CAR).

84. The engineered T cell of claim 81, wherein the engineered T cell is a gamma-delta T cell, a mucosal associated invariant T (MAIT) T cell, a natural killer (NK) cell, a natural killer T (NKT) cell, or a combination thereof.

85. The engineered T cell of claim 81, wherein the engineered T cell is a CD8⁺ T cell.

86. The engineered T cell of claim 81, wherein the engineered T cell is a CD4⁺ T cell.

87. The engineered T cell of claim 81, wherein the engineered T cell is human.

88. An engineered T cell expressing a transgene encoding a B-cell lymphoma-extra large (Bcl-XL), wherein the engineered T cell does not express cyclin-dependent kinase inhibitor 2A (CDKN2A), cyclin-dependent kinase inhibitor 2B (CDKN2B), S-methyl-5′-thioadenosine phosphorylase (MTAP), and/or phosphatase and tensin homolog (PTEN).

89. The engineered T cell of claim 88, wherein the engineered T cell does not express of one or more endogenous immune related genes in the primary immune cells.

90. The engineered T cell of claim 89, wherein the endogenous immune related gene is beta-2 microglobulin (B2M), or T-cell receptor α constant (TRAC).

91. The engineered T cell of claim 88, wherein the engineered T cell does not express cluster of differentiation 38 (CD38).

92. The engineered T cell of claim 88 further comprising a polynucleotide that encodes a chimeric antigen receptor (CAR).

93. The engineered T cell of claim 88, wherein the engineered T cell is a CD8⁺ T cell, a CD4⁺ T cell, a delta gamma T cell, a mucosal associated invariant T (MAIT) T cell, a natural killer (NK) T cell, or a combination thereof.

94. The engineered T cell of claim 88, wherein the engineered T cell is a CD8⁺ T cell.

95. The engineered T cell of claim 88, wherein the engineered T cell is a CD4⁺ T cell.

96. The engineered T cell of claim 88, wherein the engineered T cell is human.
